SEQUENCE LISTING (72 sequences)

SEQ ID NO: 1 - protein, 451 aa, Artificial Sequence
  Synthetic sequence
EVQLVESGGGLVQPGGSLRLSCAASGFTFSSYYIHWVRQAPGKGLEWVAAISSSSGSTYYADSVKGRFTISADTSKNTAYLQMNSLRAEDTAVYYCARERYFYGDFVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 10 aa, Artificial Sequence
  Synthetic sequence
GFTFSSYYIH

SEQ ID NO: 3 - protein, 14 aa, Artificial Sequence
  Synthetic sequence
AISSSSGSTYYADS

SEQ ID NO: 4 - protein, 19 aa, Artificial Sequence
  Synthetic sequence
CARERYFYGDFVFDYWGQG

SEQ ID NO: 5 - protein, 214 aa, Artificial Sequence
  Synthetic sequence
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 11 aa, Artificial Sequence
  Synthetic sequence
RASQSVSSAVA

SEQ ID NO: 7 - protein, 7 aa, Artificial Sequence
  Synthetic sequence
SASSLYS

SEQ ID NO: 8 - protein, 6 aa, Artificial Sequence
  Synthetic sequence
YYSSPL

SEQ ID NO: 9 - protein, 451 aa, Artificial Sequence
  Synthetic sequence
EVQLVESGGGLVQPGGSLRLSCAASGFTFSHHYIHWVRQAPGKGLEWVAAISTGGGTTYYADSVKGRFTISADTSKNTAYLQMNSLRAEDTAVYYCARERYFNGDFIYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 10 aa, Artificial Sequence
  Synthetic sequence
GFTFSHHYIH

SEQ ID NO: 11 - protein, 14 aa, Artificial Sequence
  Synthetic sequence
AISTGGGTTYYADS

SEQ ID NO: 12 - protein, 19 aa, Artificial Sequence
  Synthetic sequence
CARERYFNGDFIYDYWGQG

SEQ ID NO: 13 - protein, 214 aa, Artificial Sequence
  Synthetic sequence
DIQMTQSPSSLSASVGDRVTITCRASQSVSSAVAWYQQKPGKAPKLLIYSASSLYSGVPSRFSGSRSGTDFTLTISSLQPEDFATYYCQQYYSTPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 11 aa, Artificial Sequence
  Synthetic sequence
RASQSVSSAVA

SEQ ID NO: 15 - protein, 7 aa, Artificial Sequence
  Synthetic sequence
SASSLYS

SEQ ID NO: 16 - protein, 6 aa, Artificial Sequence
  Synthetic sequence
YYSTPL

SEQ ID NO: 17 - protein, 451 aa, Artificial Sequence
  Synthetic sequence
EVQLVESGGGLVQPGGSLRLSCAASGFTFSSYYIHWVRQAPGKGLEWVAAISSSSGSTYYADSVKGRFTISADTSKNTAYLQMNSLRAEDTAVYYCAREVRIHGDFVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 10 aa, Artificial Sequence
  Synthetic sequence
GFTFSSYYIH

SEQ ID NO: 19 - protein, 14 aa, Artificial Sequence
  Synthetic sequence
AISSSSGSTYYADS

SEQ ID NO: 20 - protein, 19 aa, Artificial Sequence
  Synthetic sequence
CAREVRIHGDFVLDYWGQG

SEQ ID NO: 21 - protein, 214 aa, Artificial Sequence; identical to SEQ ID NO: 5
  Synthetic sequence

SEQ ID NO: 22 - protein, 11 aa, Artificial Sequence
  Synthetic sequence
RASQSVSSAVA

SEQ ID NO: 23 - protein, 7 aa, Artificial Sequence
  Synthetic sequence
SASSLYS

SEQ ID NO: 24 - protein, 6 aa, Artificial Sequence
  Synthetic sequence
YYSSPL

SEQ ID NO: 25 - protein, 451 aa, Artificial Sequence
  Synthetic sequence
EVQLVESGGGLVQPGGSLRLSCAASGFTFSSYYIHWVRQAPGKGLEWVAAISSSSGSTYYADSVKGRFTISADTSKNTAYLQMNSLRAEDTAVYYCAREARFHGDFILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 10 aa, Artificial Sequence
  Synthetic sequence
GFTFSSYYIH

SEQ ID NO: 27 - protein, 15 aa, Artificial Sequence
  Synthetic sequence
AAISSSSGSTYYADS

SEQ ID NO: 28 - protein, 19 aa, Artificial Sequence
  Synthetic sequence
CAREARFHGDFILDYWGQG

SEQ ID NO: 29 - protein, 214 aa, Artificial Sequence
  Synthetic sequence
DIQMTQSPSSLSASVGDRVTITCRASQSVSSAVAWYQQKPGKAPKLLIYSASSLYSGVPSRFSGSRSGTDFTLTISSLQPEDFATYYCQQYYITPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 11 aa, Artificial Sequence
  Synthetic sequence
RASQSVSSAVA

SEQ ID NO: 31 - protein, 7 aa, Artificial Sequence
  Synthetic sequence
SASSLYS

SEQ ID NO: 32 - protein, 6 aa, Artificial Sequence
  Synthetic sequence
YYITPL

SEQ ID NO: 33 - protein, 455 aa, Artificial Sequence
  Synthetic sequence
EVQLVESGGGLVQPGGSLRLSCAASGFTFSSYYIHWVRQAPGKGLEWVAAISSSSGSTYYADSVKGRFTISADTSKNTAYLQMNSLRAEDTAVYYCAREDWHRYERRFYEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 10 aa, Artificial Sequence
  Synthetic sequence
GFTFSSYYIH

SEQ ID NO: 35 - protein, 15 aa, Artificial Sequence
  Synthetic sequence
AAISSSSGSTYYADS

SEQ ID NO: 36 - protein, 23 aa, Artificial Sequence
  Synthetic sequence
CAREDWHRYERRFYEGFDYWGQG

SEQ ID NO: 37 - protein, 214 aa, Artificial Sequence; identical to SEQ ID NO: 5
  Synthetic sequence

SEQ ID NO: 38 - protein, 11 aa, Artificial Sequence
  Synthetic sequence
RASQSVSSAVA

SEQ ID NO: 39 - protein, 7 aa, Artificial Sequence
  Synthetic sequence
SASSLYS

SEQ ID NO: 40 - protein, 6 aa, Artificial Sequence
  Synthetic sequence
YYSSPL

SEQ ID NO: 41 - protein, 451 aa, Artificial Sequence
  Synthetic sequence
EVQLVESGGGLVQPGGSLRLSCAASGFTFSSYYIHWVRQAPGKGLEWVAAISSSSGSTYYADSVKGRFTISADTSKNTAYLQMNSLRAEDTAVYYCAREDYYWGDFVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 10 aa, Artificial Sequence
  Synthetic sequence
GFTFSSYYIH

SEQ ID NO: 43 - protein, 15 aa, Artificial Sequence
  Synthetic sequence
AAISSSSGSTYYADS

SEQ ID NO: 44 - protein, 19 aa, Artificial Sequence
  Synthetic sequence
CAREDYYWGDFVFDYWGQG

SEQ ID NO: 45 - protein, 214 aa, Artificial Sequence; identical to SEQ ID NO: 5
  Synthetic sequence

SEQ ID NO: 46 - protein, 11 aa, Artificial Sequence
  Synthetic sequence
RASQSVSSAVA

SEQ ID NO: 47 - protein, 7 aa, Artificial Sequence
  Synthetic sequence
SASSLYS

SEQ ID NO: 48 - protein, 6 aa, Artificial Sequence
  Synthetic sequence
YYSSPL

SEQ ID NO: 49 - protein, 449 aa, Artificial Sequence
  Synthetic sequence
EVQLVESGGGLVQPGGSLRLSCAASGFTFTDYDIHWVRQAPGKGLEWVAGWINPYNGYTDYADSVKGRFTISADTSKNTAYLQMNSLRAEDTAVYYCARPLSWALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 10 aa, Artificial Sequence
  Synthetic sequence
GFTFTDYDIH

SEQ ID NO: 51 - protein, 16 aa, Artificial Sequence
  Synthetic sequence
AGWINPYNGYTDYADS

SEQ ID NO: 52 - protein, 15 aa, Artificial Sequence
  Synthetic sequence
CARPLSWALDYWGQG

SEQ ID NO: 53 - protein, 214 aa, Artificial Sequence
  Synthetic sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 11 aa, Artificial Sequence
  Synthetic sequence
RASQDVNTAVA

SEQ ID NO: 55 - protein, 7 aa, Artificial Sequence
  Synthetic sequence
SASFLYS

SEQ ID NO: 56 - protein, 6 aa, Artificial Sequence
  Synthetic sequence
HYTTPP

SEQ ID NO: 57 - protein, 450 aa, Artificial Sequence
  Synthetic sequence
EVQLVESGGGLVQPGGSLRLSCAASGFTFTDYDIHWVRQAPGKGLEWVAYIRPSGSKTSYADSVKGRFTISADTSKNTAYLQMNSLRAEDTAVYYCARKVYARSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 10 aa, Artificial Sequence
  Synthetic sequence
GFTFSSYYIH

SEQ ID NO: 59 - protein, 15 aa, Artificial Sequence
  Synthetic sequence
AYIRPSGSKTSYADS

SEQ ID NO: 60 - protein, 17 aa, Artificial Sequence
  Synthetic sequence
CARKVYARSGLDYWGQG

SEQ ID NO: 61 - protein, 214 aa, Artificial Sequence
  Synthetic sequence
DIQMTQSPSSLSASVGDRVTITCRASQSVSSAVAWYQQKPGKAPKLLIYSASSLYSGVPSRFSGSRSGTDFTLTISSLQPEDFATYYCQQGESTPYTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 11 aa, Artificial Sequence
  Synthetic sequence
RASQSVSSAVA

SEQ ID NO: 63 - protein, 7 aa, Artificial Sequence
  Synthetic sequence
SASSLYS

SEQ ID NO: 64 - protein, 6 aa, Artificial Sequence
  Synthetic sequence
GESTPY

SEQ ID NO: 65 - protein, 448 aa, Artificial Sequence
  Synthetic sequence
EVQLVESGGGLVQPGGSLRLSCAASGFTFTDYDIHWVRQAPGKGLEWVAYIYSRGTVTSYADSVKGRFTISADTSKNTAYLQMNSLRAEDTAVYYCARGAGGVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 10 aa, Artificial Sequence
  Synthetic sequence
GFTFSSYGIH

SEQ ID NO: 67 - protein, 15 aa, Artificial Sequence
  Synthetic sequence
AYIYSRGTVTSYADS

SEQ ID NO: 68 - protein, 15 aa, Artificial Sequence
  Synthetic sequence
CARGAGGVMDYWGQG

SEQ ID NO: 69 - protein, 214 aa, Artificial Sequence
  Synthetic sequence
DIQMTQSPSSLSASVGDRVTITCRASQSVSSAVAWYQQKPGKAPKLLIYSASSLYSGVPSRFSGSRSGTDFTLTISSLQPEDFATYYCQQAKSSLRTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 11 aa, Artificial Sequence
  Synthetic sequence
RASQSVSSAVA

SEQ ID NO: 71 - protein, 7 aa, Artificial Sequence
  Synthetic sequence
SASSLYS

SEQ ID NO: 72 - protein, 6 aa, Artificial Sequence
  Synthetic sequence
AKSSLR

(19) United States
(12) Patent Application Publication
(10) Pub. No.: US 2022/0088193 A1
(43) Pub. Date: Mar. 24, 2022

(54) Anti-CD55 Antibodies and Related Compositions and Methods

(71) Applicant: AgonOx, Inc., Portland, OR (US)

(72) Inventors: Ryan D. Montler, Camas, WA (US); Andrew D. Weinberg, Portland, OR (US); Nicholas P. Morris, Portland, OR (US); Colin J. Thalhofer, Washougal, WA (US)

(21) Appl. No.: 17/431,107

(22) PCT Filed: Feb. 19, 2020
(86) PCT No.: PCT/US2020/018865

§ 371 Date: Aug. 13, 2021

Related U.S. Application Data
(60) Provisional application No. 62/808,146, filed on Feb. 20, 2019.

(51) Int. Cl. A61K 39/395; C07K 16/28; A61K 47/68; A61K 38/19; A61P 35/00

(52) U.S. Cl. CPC A61K 39/3955; C07K 16/2896; A61K 47/6849; A61K 38/19; A61P 35/00; C07K 2317/56; A61K 2039/55561; C07K 2317/24; C07K 2317/55; C07K 2317/622; C07K 2317/31; C07K 2319/33; C07K 2317/565

(57) ABSTRACT

Provided are antibodies that specifically bind CD55. Nucleic acids that encode one or both of the variable chain polypeptides of an antibody of the present disclosure are also provided, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual having a cell proliferative disorder a therapeutically effective amount of an antibody of the present disclosure, where the antibody is administered to the individual to enhance a T cell response to abnormally proliferating cells of the cell proliferative disorder.

### CROSS-REFERENCE TO RELATED APPLICATIONS

This application claims the benefit of U.S. Provisional Patent Application No. 62/808,146, filed Feb. 20, 2019, which application is incorporated herein by reference in its entirety.

### INTRODUCTION

T cell-mediated immunity includes multiple sequential steps involving the clonal selection of antigen-specific cells, their activation and proliferation in secondary lymphoid tissues, their trafficking to sites of antigen and inflammation, the execution of direct effector functions and the provision of help (through cytokines and membrane ligands) for a multitude of effector immune cells. Each of these steps is regulated by counterbalancing stimulatory and inhibitory signals that fine-tune the response. Although virtually all inhibitory signals in the immune response ultimately affect intracellular signaling pathways, many are initiated through membrane receptors, the ligands of which are either membrane-bound or soluble (cytokines).

Genetic and epigenetic alterations that are characteristic of all cancers provide a diverse set of antigens that the immune system can use to distinguish tumor cells from their normal counterparts. In the case of T cells, the ultimate amplitude and quality of the response, which is initiated through antigen recognition by the T cell receptor (TCR), is regulated by a balance between co-stimulatory and inhibitory signals (that is, immune checkpoints). Under normal physiological conditions, immune checkpoints are crucial for the maintenance of self-tolerance (that is, the prevention of autoimmunity) and also to protect tissues from damage when the immune system is responding to pathogenic infection.

The expression of immune-checkpoint proteins can be dysregulated by tumors as an important immune resistance mechanism. T cells have been the major focus of efforts to therapeutically manipulate endogenous anti-tumor immunity owing to: their capacity for the selective recognition of peptides derived from proteins in all cellular compartments; their capacity to directly recognize and kill antigen-expressing cells (by CD8+ effector T cells—also known as cytotoxic T lymphocytes (CTLs)); and their ability to orchestrate diverse immune responses (by CD4+ helper T cells), which integrates adaptive and innate effector mechanisms. Thus, agonists of co-stimulatory receptors or antagonists of inhibitory signals, both of which result in the amplification of antigen-specific T cell responses, are agents of particular clinical interest.

CD55 (UniProtKB—P08174 (human); also known as Decay-Accelerating Factor, or “DAF”) is a 70 kDa membrane protein that attaches to the cell membrane via a glycophosphatidylinositol (GPI) anchor. This protein contains four complement control protein (CCP) repeats with a single N-linked glycan positioned between CCP1 and CCP2. CCP2, CCP3, CCP4 and three consecutive lysine residues in a positively charged pocket between CCP2 and CCP3 are involved in its inhibition of the alternate complement pathway. CCP2 and CCP3 alone are involved in its inhibition of the classical pathway.

CD55 recognizes C4b and C3b fragments that condense with cell-surface hydroxyl or amino groups when nascent C4b and C3b are locally generated during C4 and C3 activation. Interaction of CD55 with cell-associated C4b and C3b polypeptides interferes with their ability to catalyze the conversion of C2 and factor B to enzymatically active C2a and Bb, thereby preventing the formation of C4b2a and C3bBb, the amplification convertases of the complement cascade. CD55 further acts as a receptor for coxsackievirus A21, coxsackieviruses B1, B3 and B5, and human echoviruses 6, 7, 11, 12, 20 and 21.

CD55 overexpression has been observed on a variety of human tumor tissues including lung adenocarcinomas and lung squamous cell carcinomas, e.g., see Niehans et al., Am. J. Path. 149:129-142 (1996). U.S. Pat. No. 7,288,249 describes the administration of anti-CD55 antibodies to a subpopulation of cancer patients identified as overexpressing CD55 or expressing a cancer-related variant of CD55. According to the '249 patent, the antibodies administered to the subpopulation of cancer patients find use for antibody-dependent cellular cytotoxicity (ADCC) and may be conjugated to a cytotoxic agent for enhanced killing of cancer cells overexpressing CD55.

### SUMMARY

Provided are antibodies that specifically bind CD55. Nucleic acids that encode one or both of the variable chain polypeptides of an antibody of the present disclosure are also provided, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual having a cell proliferative disorder a therapeutically effective amount of an antibody of the present disclosure, where the antibody is administered to the individual to enhance a T cell response to abnormally proliferating cells of the cell proliferative disorder.

### BRIEF DESCRIPTION OF THE FIGURES

FIG. 1: Testing of antibodies for the ability to co-stimulate human CD4 T-cell proliferation in combination with subthreshold anti-CD3 antibody where all antibodies are plate-bound. Panel A: costimulatory activity of antibodies P5-1, P5-2, P5-3, P5-10B P5-34, P5-37. Panel B: Costimulatory activity of P5-1, P5-1J, P5-2-2A, P5-4. Solid symbols represent the antibodies that recognize only human CD55, and open symbols represent antibodies that recognized both mouse and human CD55. The commercially available anti-CD55 antibody, JS11, was included in panel B as a positive control. Each antibody co-stimulated CD4 T-cell proliferation to varying degrees. The differential biologic effectiveness of the P5-1, P5-1J, P5-2-2A and P5-4 antibodies in this assay was not reflected by their relative affinity (see FIG. 2, panel A).

FIG. 2: ELISA data for antibodies that bind both mouse and human CD55 (P5-1, P5-1J, P5-2-2A and P5-4). Relative affinities for human CD55 and mouse CD55 are shown in panels A and B, respectively. EC₅₀ values were obtained by fitting to a sigmoid non-linear equation, using PRISM. These human antibodies contained mouse IgG2A Fc domains for use in in vivo mouse tumor models and were detected by HRP-anti-mouse secondary antibodies. The antibodies tested had very similar affinities for human CD55 (about 4 ng/ml) but varied affinities for mouse CD55 (about 3-22 ng/ml).

FIG. 3: Competition ELISA data for comparing the epitopes of the mouse+human CD55 binding antibodies. Since P5-1 and P5-1J share their epitope, P5-1 was not examined. Plate-bound CD55 was incubated with unlabeled P5-1J, P5-2-2A and P5-4 antibodies at 1 μg/ml. The plates were washed and then incubated with the indicated concentrations of biotinylated P5-1J. Streptavidin-HRP was used for detection of bound P5-1J. The EC₅₀ values are shown below the graph. P5-2-2A and P5-4 blocked biotinylated P5-1J binding (20-fold shift in EC₅₀) but not to the extent that unlabeled P5-1J blocked it (100-fold shift in EC₅₀). The data suggests that the P5-1J, P5-2-2A and P5-4 epitopes are the same, overlapping, or near enough to cause steric interference.

FIG. 4: Competitive cell binding data for comparison of the epitopes of the mouse+human CD55 binding antibodies. Human peripheral blood mononuclear cells (PBMCs) were incubated with the concentrations of P5 antibodies indicated on the abscissa, washed and then labeled with biotinylated P5-1J followed by Streptavidin-PE and finally analyzed by flow cytometry. The mean fluorescence intensity (MFI) is a measure of the amount of biotinylated P5-1J bound to the cells. Binding to cells is more biologically relevant than binding to purified protein. Both P5-2-2A and P5-4 significantly reduced the binding of P5-1J to CD55, this time on cells and consistent with the ELISA results indicating either identity or close proximity of the P5-1J, P5-2-2A and P5-4 antibody epitopes on CD55.

FIG. 5: Effect of anti-CD55 antibodies on tumor growth in vivo. Mice were inoculated s.c. with 4e5 MCA 205 tumor cells on Day 0. Tumor sizes were measured 2-3 times per week. Mice were treated on days 9, 12 and 15 with either 200 μg of hamster IgG control antibody or 200 μg anti-PD1 i.p. concurrently with either 200 μg of mouse IgG control antibody or antibody P5-1J i.p.. Single agent anti-PD-1-treated mice had 2/10 mice reject their tumors. The anti-PD-1 and P5-1J combination treatment group had 5/10 mice reject their tumors. Spider plots are shown for each treatment group, where each line represents a single mouse. A survival curve is shown in the lower right corner.

FIG. 6: Effect of anti-CD55 antibodies on tumor growth in vivo. Mice were inoculated s.c. with 4e5 MCA 205 tumor cells on Day 0. Tumor sizes were measured 2-3 times per week. Mice were treated on days 12, 14 and 16 with either 10 μl of saline i.t. or 30 μg of CpG i.t. alone or concurrently with 200 μg of antibody P5-1J i.p.. Single therapeutic agent CpG-treated mice had 1/5 mice reject their tumors. The CpG and P5-1J combination treatment group had 2/5 mice reject their tumors. Spider plots are shown for each treatment group, each line represents a single mouse. A survival curve is shown in the lower right corner.

FIG. 7: Effect of anti-CD55 antibodies on tumor growth in vivo. Mice were inoculated s.c. with 4e5 MCA 205 tumor cells on Day 0. Tumor sizes were measured 2-3 times per week. Mice were treated on days 7, 9 and 11 with either 200 μg of Isotype control antibody or 250 μg anti-OX40 i.p. concurrently with either 200 μg of mouse IgG control antibody or antibody P5-1, P5-2-2a, or P5-4 i.p.. None of the isotype control antibody-treated mice rejected their tumor. Single therapeutic agent anti-OX40-treated mice had 0/5 mice reject their tumors. The P5-1 and anti-OX40 combination treatment group had 2/10 mice reject their tumors. The P5-2-2a and anti-OX40 combination treatment group had 3/10 mice reject their tumors. The P5-4 and anti-OX40 combination treatment group had 6/10 mice reject their tumors. Spider plots are shown for each treatment group, each line represents a single mouse. A survival curve is shown in the lower right corner.

FIG. 8: Effect of anti-CD55 antibodies on tumor growth in vivo. Mice were inoculated s.c. with 4e5 MCA 205 tumor cells on Day 0. Tumor sizes were measured 2-3 times per week. Mice were treated on days 6, 8 and 10 with either 200 μg of Isotype control antibody or 200 ug anti-PD-1 i.p. concurrently with either 200 μg of mouse IgG control antibody or antibody P5-1 or P5-4 i.p.. None of the isotype control antibody-treated mice rejected their tumor. Single therapeutic agent anti-PD-1-treated mice had 2/10 mice reject their tumors. The P5-1 and anti-PD-1 combination treatment group had 3/10 mice reject their tumors. The P5-4 and anti-PD-1 combination treatment group had 4/10 mice reject their tumors. Spider plots are shown for each treatment group (except the Isotype only group), each line represents a single mouse. A survival curve is shown in the lower right corner.

### DETAILED DESCRIPTION

As summarized above, provided are antibodies that specifically bind CD55. Nucleic acids that encode one or both of the variable chain polypeptides of an antibody of the present disclosure are also provided, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual having a cell proliferative disorder a therapeutically effective amount of an antibody of the present disclosure, where the antibody is administered to the individual to enhance a T cell response to abnormally proliferating cells of the cell proliferative disorder.

Before the antibodies, compositions and methods of the present disclosure are described in greater detail, it is to be understood that the antibodies, compositions and methods are not limited to particular embodiments described, as such may, of course, vary. It is also to be understood that the terminology used herein is for the purpose of describing particular embodiments only, and is not intended to be limiting, since the scope of the antibodies, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies, compositions and methods. The upper and lower limits of these smaller ranges may independently be included in the smaller ranges and are also encompassed within the antibodies, compositions and methods, subject to any specifically excluded limit in the stated range. Where the stated range includes one or both of the limits, ranges excluding either or both of those included limits are also included in the antibodies, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies, compositions and methods belong. Although any antibodies, compositions and methods similar or equivalent to those described herein can also be used in the practice or testing of the antibodies, compositions and methods, representative illustrative antibodies, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies, compositions and methods, which are, for clarity, described in the context of separate embodiments, may also be provided in combination in a single embodiment. Conversely, various features of the antibodies, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present antibodies, compositions and methods. Any recited method can be carried out in the order of events recited or in any other order that is logically possible.

### Antibodies

As summarized above, the present disclosure provides anti-CD55 antibodies. As demonstrated in the Experimental section below, human antibodies that bind both mouse and human CD55 were identified, and such antibodies were found to co-stimulate human CD4 T-cell proliferation. Moreover, the antibodies were found to enhance the inhibition of tumor growth in vivo, e.g., as compared to inhibition of tumor growth by a T cell activator alone or an innate immune system stimulator alone. The antibodies find use in a variety of research and therapeutic applications, including for performing any of the methods described in International Patent Application No. PCT/US2018/047356, the disclosure of which is incorporated herein by reference in its entirety for all purposes.

In certain embodiments, an antibody of the present disclosure specifically binds to CD55 and is an agonist of CD55. An example assay that may be employed to determine whether an anti-CD55 antibody is a CD55 agonist is an assay in which co-stimulation of CD4 T cells is assessed for a plate-bound anti-CD55 antibody and the corresponding soluble anti-CD55 antibody. Co-stimulation by the anti-CD55 antibody when plate-bound but not when soluble (or greater co-stimulation by the anti-CD55 antibody when plate-bound as compared to when soluble) indicates that the anti-CD55 antibody is an agonist, e.g., because the plate-bound anti-CD55 antibody is able to concentrate CD55 molecules on the surface of the cell, thereby facilitating signaling through CD55. Further details may be found in International Patent Application No. PCT/US2018/047356, the disclosure of which is incorporated herein by reference in its entirety for all purposes.

According to some embodiments, an antibody of the present disclosure specifically binds to CD55, is an agonist of CD55, and enhances the inhibition of tumor growth in vivo, e.g., as compared to inhibition of tumor growth by a T cell activator in the absence of the anti-CD55 antibody. Such enhancement may be assessed, e.g., using an in vivo mouse tumor model (e.g., using MCA 205 tumor cells or other suitable tumor cells), or the like. Non-limiting examples of such enhancement are provided in the Experimental section below. In some embodiments, the anti-CD55 antibody enhances the inhibition of tumor growth in vivo by 10% or greater, 20% or greater, 30% or greater, 40% or greater, 50% or greater, 60% or greater, 70% or greater, 80% or greater, 90% or greater, 100% or greater, 110% or greater, 120% or greater, 130% or greater, 140% or greater, 150% or greater, 160% or greater, 170% or greater, 180% or greater, 190% or greater, or 200% or greater, as compared to inhibition of tumor growth by a T cell activator in the absence of the anti-CD55 antibody.

According to some embodiments, an antibody of the present disclosure specifically binds to CD55, is an agonist of CD55, and enhances the inhibition of tumor growth in vivo, e.g., as compared to inhibition of tumor growth by an innate immune system stimulator in the absence of the anti-CD55 antibody. Such enhancement may be assessed, e.g., using an in vivo mouse tumor model (e.g., using MCA 205 tumor cells or other suitable tumor cells), or the like. A non-limiting example of such enhancement is provided in the Experimental section below. In some embodiments, the anti-CD55 antibody enhances the inhibition of tumor growth in vivo by 10% or greater, 20% or greater, 30% or greater, 40% or greater, 50% or greater, 60% or greater, 70% or greater, 80% or greater, 90% or greater, 100% or greater, 110% or greater, 120% or greater, 130% or greater, 140% or greater, 150% or greater, 160% or greater, 170% or greater, 180% or greater, 190% or greater, or 200% or greater, as compared to inhibition of tumor growth by an innate immune system stimulator in the absence of the anti-CD55 antibody.

In certain embodiments, an antibody of the present disclosure specifically binds to CD55 and competes for binding to CD55 with an antibody having one, two, three, four, five, or all six complementarity determining regions (CDRs) of one or more of the anti-CD55 antibodies designated herein as P5-1, P5-1J, P5-2, P5-2-2A, P5-3, P5-4, P5-10B, P5-34, and P5-37. The amino acid sequences of the variable heavy chain (V_{H}) polypeptides, the variable light chain (V_{L}) polypeptides, and the CDRs of the P5-1, P5-1J, P5-2, P5-2-2A, P5-3, P5-4, P5-10B, P5-34, and P5-37 antibodies are provided in Table 1 below.

TABLE 1
Amino Acid Sequences of Example Anti-CD55 Antibodies

| | |
|---|---|
| P5-1 V_{H} | EVQLVESGGGLVQPGGSLRLSCAASGFTFSSYYIHWVRQAPGKGLEWVAAISSSS |
| SEQ ID NO: 1 | GSTYYADSVKGRFTISADTSKNTAYLQMNSLRAEDTAVYYCARERYFYGDFVFDY |
| | WGQGTLVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEALHNHYTQ |
| | KSLSLSPGK |
| P5-1 V_{H} CDR1 | GFTFSSYYIH |
| SEQ ID NO: 2 | |
| P5-1 V_{H} CDR2 | AISSSSGSTYYADS |
| SEQ ID NO: 3 | |
| P5-1 V_{H} CDR3 | CARERYFYGDFVFDYWGQG |
| SEQ ID NO: 4 | |
| P5-1 V_{L} | DIQMTQSPSSLSASVGDRVTITCRASQSVSSAVAWYQQKPGKAPKLLIYSASSLYS |
| SEQ ID NO: 5 | GVPSRFSGSRSGTDFTLTISSLQPEDFATYYCQQYYSSPLTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-1 V_{L} CDR1 | RASQSVSSAVA |
| SEQ ID NO: 6 | |
| P5-1 V_{L} CDR2 | SASSLYS |
| SEQ ID NO: 7 | |
| P5-1 V_{L} CDR3 | YYSSPL |
| SEQ ID NO: 8 | |
| P5-1J V_{H} | EVQLVESGGGLVQPGGSLRLSCAASGFTFSHHYIHWVRQAPGKGLEWVAAISTGG |
| SEQ ID NO: 9 | GTTYYADSVKGRFTISADTSKNTAYLQMNSLRAEDTAVYYCARERYFNGDFIYDYW |
| | GQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQK |
| | SLSLSPGK |
| P5-1J V_{H} | GFTFSHHYIH |
| CDR1 | |
| SEQ ID NO: 10 | |
| P5-1J V_{H} | AISTGGGTTYYADS |
| CDR2 | |
| SEQ ID NO: 11 | |
| P5-1J V_{H} | CARERYFNGDFIYDYWGQG |
| CDR3 | |
| SEQ ID NO: 12 | |
| P5-1J V_{L} | DIQMTQSPSSLSASVGDRVTITCRASQSVSSAVAWYQQKPGKAPKLLIYSASSLYS |
| SEQ ID NO: 13 | GVPSRFSGSRSGTDFTLTISSLQPEDFATYYCQQYYSTPLTFGQGTKVEIKRTVAAP |
| | SVFIFPPSDSQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| P5-1J V_{L} CDR1 | RASQSVSSAVA |
| SEQ ID NO: 14 | |
| P5-1J V_{L} CDR2 | SASSLYS |
| SEQ ID NO: 15 | |
| P5-1J V_{L} CDR3 | YYSTPL |
| SEQ ID NO: 16 | |
| P5-2 V_{H} | EVQLVESGGGLVQPGGSLRLSCAASGFTFSSYYIHWVRQAPGKGLEWVAAISSSS |
| SEQ ID NO: 17 | GSTYYADSVKGRFTISADTSKNTAYLQMNSLRAEDTAVYYCAREVRIHGDFVLDYW |
| | GQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQK |
| | SLSLSPGK |
| P5-2 V_{H} CDR1 | GFTFSSYYIH |
| SEQ ID NO: 18 | |
| P5-2 V_{H} CDR2 | AISSSSGSTYYADS |
| SEQ ID NO: 19 | |
| P5-2 V_{H} CDR3 | CAREVRIHGDFVLDYWGQG |
| SEQ ID NO: 20 | |
| P5-2 V_{L} | DIQMTQSPSSLSASVGDRVTITCRASQSVSSAVAWYQQKPGKAPKLLIYSASSLYS |
| SEQ ID NO: 21 | GVPSRFSGSRSGTDFTLTISSLQPEDFATYYCQQYYSSPLTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-2 V_{L} CDR1 | RASQSVSSAVA |
| SEQ ID NO: 22 | |
| P5-2 V_{L} CDR2 | SASSLYS |
| SEQ ID NO: 23 | |
| P5-2 V_{L} CD R3 | YYSSPL |
| SEQ ID NO: 24 | |
| P5-2-2A V_{H} | EVQLVESGGGLVQPGGSLRLSCAASGFTFSSYYIHWVRQAPGKGLEWVAAISSSS |
| SEQ ID NO: 25 | GSTYYADSVKGRFTISADTSKNTAYLQMNSLRAEDTAVYYCAREARFHGDFILDYW |
| | GQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQK |
| | SLSLSPGK |
| P5-2-2A V_{H} | GFTFSSYYIH |
| CDR1 | |
| SEQ ID NO: 26 | |
| P5-2-2A V_{H} | AAISSSSGSTYYADS |
| CDR2 | |
| SEQ ID NO: 27 | |
| P5-2-2A V_{H} | CAREARFHGDFILDYWGQG |
| CD R3 | |
| SEQ ID NO: 28 | |
| P5-2-2A V_{L} | DIQMTQSPSSLSASVGDRVTITCRASQSVSSAVAWYQQKPGKAPKLLIYSASSLYS |
| SEQ ID NO: 29 | GVPSRFSGSRSGTDFTLTISSLQPEDFATYYCQQYYITPLTFGQGTKVEIKRTVAAP |
| | SVFIFPPSDSQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| P5-2-2A V_{L} | RASQSVSSAVA |
| CDR1 | |
| SEQ ID NO: 30 | |
| P5-2-2A V_{L} | SASSLYS |
| CDR2 | |
| SEQ ID NO: 31 | |
| P5-2-2A V_{L} | YYITPL |
| CD R3 | |
| SEQ ID NO: 32 | |
| P5-3 V_{H} | EVQLVESGGGLVQPGGSLRLSCAASGFTFSSYYIHWVRQAPGKGLEWVAAISSSS |
| SEQ ID NO: 33 | GSTYYADSVKGRFTISADTSKNTAYLQMNSLRAEDTAVYYCAREDWHRYERRFYE |
| | GFDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHN |
| | HYTQKSLSLSPGK |
| P5-3 V_{H} CDR1 | GFTFSSYYIH |
| SEQ ID NO: 34 | |
| P5-3 V_{H} CDR2 | AAISSSSGSTYYADS |
| SEQ ID NO: 35 | |
| P5-3 V_{H} CDR3 | CAREDWHRYERRFYEGFDYWGQG |
| SEQ ID NO: 36 | |
| P5-3 V_{L} | DIQMTQSPSSLSASVGDRVTITCRASQSVSSAVAWYQQKPGKAPKLLIYSASSLYS |
| SEQ ID NO: 37 | GVPSRFSGSRSGTDFTLTISSLQPEDFATYYCQQYYSSPLTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-3 V_{L} CDR1 | RASQSVSSAVA |
| SEQ ID NO: 38 | |
| P5-3 V_{L} CDR2 | SASSLYS |
| SEQ ID NO: 39 | |
| P5-3 V_{L} CDR3 | YYSSPL |
| SEQ ID NO: 40 | |
| P5-4 V_{H} | EVQLVESGGGLVQPGGSLRLSCAASGFTFSSYYIHWVRQAPGKGLEWVAAISSSS |
| SEQ ID NO: 41 | GSTYYADSVKGRFTISADTSKNTAYLQMNSLRAEDTAVYYCAREDYYWGDFVFDY |
| | WGQGTLVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEALHNHYTQ |
| | KSLSLSPGK |
| P5-4 V_{H} CDR1 | GFTFSSYYIH |
| SEQ ID NO: 42 | |
| P5-4 V_{H} CDR2 | AAISSSSGSTYYADS |
| SEQ ID NO: 43 | |
| P5-4 V_{H} CDR3 | CAREDYYWGDFVFDYWGQG |
| SEQ ID NO: 44 | |
| P5-4 V_{L} | DIQMTQSPSSLSASVGDRVTITCRASQSVSSAVAWYQQKPGKAPKLLIYSASSLYS |
| SEQ ID NO: 45 | GVPSRFSGSRSGTDFTLTISSLQPEDFATYYCQQYYSSPLTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-4 V_{L} CDR1 | RASQSVSSAVA |
| SEQ ID NO: 46 | |
| P5-4 V_{L} CDR2 | SASSLYS |
| SEQ ID NO: 47 | |
| P5-4 V_{L} CDR3 | YYSSPL |
| SEQ ID NO: 48 | |
| P5-10B V_{H} | EVQLVESGGGLVQPGGSLRLSCAASGFTFTDYDIHWVRQAPGKGLEWVAGWINP |
| SEQ ID NO: 49 | YNGYTDYADSVKGRFTISADTSKNTAYLQMNSLRAEDTAVYYCARPLSWALDYWG |
| | QGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPPKS |
| | CDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| P5-10B V_{H} | GFTFTDYDIH |
| CDR1 | |
| SEQ ID NO: 50 | |
| P5-10B V_{H} | AGWINPYNGYTDYADS |
| CD R2 | |
| SEQ ID NO: 51 | |
| P5-10B V_{H} | CARPLSWALDYWGQG |
| CD R3 | |
| SEQ ID NO: 52 | |
| P5-10B V_{L} | DIQMTQSPSSLSASVGDRVTITCRASQDVNTAVAWYQQKPGKAPKLLIYSASFLYS |
| SEQ ID NO: 53 | GVPSRFSGSRSGTDFTLTISSLQPEDFATYYCQQHYTTPPTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-10B V_{L} | RASQDVNTAVA |
| CDR1 | |
| SEQ ID NO: 54 | |
| P5-10B V_{L} | SASFLYS |
| CD R2 | |
| SEQ ID NO: 55 | |
| P5-10B V_{L} | HYTTPP |
| CD R3 | |
| SEQ ID NO: 56 | |
| P5-34 V_{H} | EVQLVESGGGLVQPGGSLRLSCAASGFTFTDYDIHWVRQAPGKGLEWVAYIRPSG |
| SEQ ID NO: 57 | SKTSYADSVKGRFTISADTSKNTAYLQMNSLRAEDTAVYYCARKVYARSGLDYWG |
| | QGTLVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPPKS |
| | CDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| P5-34 V_{H} | GFTFSSYYIH |
| CDR1 | |
| SEQ ID NO: 58 | |
| P5-34 V_{H} | AYIRPSGSKTSYADS |
| CDR2 | |
| SEQ ID NO: 59 | |
| P5-34 V_{H} | CARKVYARSGLDYWGQG |
| CDR3 | |
| SEQ ID NO: 60 | |
| P5-34 V_{L} | DIQMTQSPSSLSASVGDRVTITCRASQSVSSAVAWYQQKPGKAPKLLIYSASSLYS |
| SEQ ID NO: 61 | GVPSRFSGSRSGTDFTLTISSLQPEDFATYYCQQGESTPYTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-34 V_{L} CDR1 | RASQSVSSAVA |
| SEQ ID NO: 62 | |
| P5-34 V_{L} CDR2 | SASSLYS |
| SEQ ID NO: 63 | |
| P5-34 V_{L} CDR3 | GESTPY |
| SEQ ID NO: 64 | |
| P5-37 V_{H} | EVQLVESGGGLVQPGGSLRLSCAASGFTFTDYDIHWVRQAPGKGLEWVAYIYSRG |
| SEQ ID NO: 65 | TVTSYADSVKGRFTISADTSKNTAYLQMNSLRAEDTAVYYCARGAGGVMDYWGQ |
| | GTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| P5-37 V_{H} | GFTFSSYGIH |
| CDR1 | |
| SEQ ID NO: 66 | |
| P5-37 V_{H} | AYIYSRGTVTSYADS |
| CDR2 | |
| SEQ ID NO: 67 | |
| P5-37 V_{H} | CARGAGGVMDYWGQG |
| CDR3 | |
| SEQ ID NO: 68 | |
| P5-37 V_{L} | DIQMTQSPSSLSASVGDRVTITCRASQSVSSAVAWYQQKPGKAPKLLIYSASSLYS |
| SEQ ID NO: 69 | GVPSRFSGSRSGTDFTLTISSLQPEDFATYYCQQAKSSLRTFGQGTKVEIKRTVAA |
| | PSVFIFPPSDSQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| P5-37 V_{L} CDR1 | RASQSVSSAVA |
| SEQ ID NO: 70 | |
| P5-34 V_{L} CDR2 | SASSLYS |
| SEQ ID NO: 71 | |
| P5-34 V_{L} CDR3 | AKSSLR |
| SEQ ID NO: 72 | |

According to some embodiments, an antibody of the present disclosure specifically binds to CD55 and competes for binding to CD55 with an antibody comprising:

a variable heavy chain (V_{H}) polypeptide comprising
a V_{H} CDR1 comprising the amino acid sequence

| | |
|---|---|
| GFTFSSYYIH, | (SEQ ID NO: 2) |

a V_{H} CDR2 comprising the amino acid sequence

| | |
|---|---|
| AISSSSGSTYYADS, | (SEQ ID NO: 3) |
| and | |

a V_{H} CDR3 comprising the amino acid sequence

| | |
|---|---|
| CARERYFYGDFVFDYWGQG; | (SEQ ID NO: 4) |
| and | |

a variable light chain (V_{L}) polypeptide comprising
a V_{L} CDR1 comprising the amino acid sequence

| | |
|---|---|
| RASQSVSSAVA, | (SEQ ID NO: 6) |

a V_{L} CDR2 comprising the amino acid sequence

| | |
|---|---|
| SASSLYS, | (SEQ ID NO: 7) |
| and | |

a V_{L} CDR3 comprising the amino acid sequence

| | |
|---|---|
| YYSSPL. | (SEQ ID NO: 8) |

In certain embodiments, an antibody of the present disclosure specifically binds to CD55 and competes for binding to CD55 with an antibody comprising:

a variable heavy chain (V_{H}) polypeptide comprising
a V_{H} CDR1 comprising the amino acid sequence

| | |
|---|---|
| GFTFSHHYIH, | (SEQ ID NO: 10) |

a V_{H} CDR2 comprising the amino acid sequence

| | |
|---|---|
| AISTGGGTTYYADS, | (SEQ ID NO: 11) |
| and | |

a V_{H} CDR3 comprising the amino acid sequence

| | |
|---|---|
| CARERYFNGDFIYDYWGQG; | (SEQ ID NO: 12) |
| and | |

a variable light chain (V_{L}) polypeptide comprising
a V_{L} CDR1 comprising the amino acid sequence

| | |
|---|---|
| RASQSVSSAVA, | (SEQ ID NO: 14) |

a V_{L} CDR2 comprising the amino acid sequence

| | |
|---|---|
| SASSLYS, | (SEQ ID NO: 15) |
| and | |

a V_{L} CDR3 comprising the amino acid sequence

| | |
|---|---|
| YYSTPL. | (SEQ ID NO: 16) |

According to some embodiments, an antibody of the present disclosure specifically binds to CD55 and competes for binding to CD55 with an antibody comprising:

a variable heavy chain (V_{H}) polypeptide comprising
a V_{H} CDR1 comprising the amino acid sequence

| | |
|---|---|
| GFTFSSYYIH, | (SEQ ID NO: 18) |

a V_{H} CDR2 comprising the amino acid sequence

| | |
|---|---|
| AISSSSGSTYYADS, | (SEQ ID NO: 19) |
| and | |

a V_{H} CDR3 comprising the amino acid sequence

| | |
|---|---|
| CAREVRIHGDFVLDYWGQG; | (SEQ ID NO: 20) |
| and | |

a variable light chain (V_{L}) polypeptide comprising
a V_{L} CDR1 comprising the amino acid sequence

| | |
|---|---|
| RASQSVSSAVA, | (SEQ ID NO: 22) |

a V_{L} CDR2 comprising the amino acid sequence

| | |
|---|---|
| SASSLYS, | (SEQ ID NO: 23) |
| and | |

a V_{L} CDR3 comprising the amino acid sequence

| | |
|---|---|
| YYSSPL. | (SEQ ID NO: 24) |

In certain embodiments, an antibody of the present disclosure specifically binds to CD55 and competes for binding to CD55 with an antibody comprising:

a variable heavy chain (V_{H}) polypeptide comprising
a V_{H} CDR1 comprising the amino acid sequence

| | |
|---|---|
| GFTFSSYYIH, | (SEQ ID NO: 26) |

a V_{H} CDR2 comprising the amino acid sequence

| | |
|---|---|
| AAISSSSGSTYYADS, | (SEQ ID NO: 27) |
| and | |

a V_{H} CDR3 comprising the amino acid sequence

| | |
|---|---|
| CAREARFHGDFILDYWGQG; | (SEQ ID NO: 28) |
| and | |

a variable light chain (V_{L}) polypeptide comprising
a V_{L} CDR1 comprising the amino acid sequence

| | |
|---|---|
| RASQSVSSAVA, | (SEQ ID NO: 30) |

a V_{L} CDR2 comprising the amino acid sequence

| | |
|---|---|
| SASSLYS, | (SEQ ID NO: 31) |
| and | |

a V_{L} CDR3 comprising the amino acid sequence

| | |
|---|---|
| YYITPL. | (SEQ ID NO: 32) |

According to some embodiments, an antibody of the present disclosure specifically binds to CD55 and competes for binding to CD55 with an antibody comprising:

a variable heavy chain (V_{H}) polypeptide comprising
a V_{H} CDR1 comprising the amino acid sequence

| | |
|---|---|
| GFTFSSYYIH, | (SEQ ID NO: 34) |

a V_{H} CDR2 comprising the amino acid sequence

| | |
|---|---|
| AAISSSSGSTYYADS, | (SEQ ID NO: 35) |
| and | |

a V_{H} CDR3 comprising the amino acid sequence

| | |
|---|---|
| CAREDWHRYERRFYEGFDYWGQG; | (SEQ ID NO: 36) |
| and | |

a variable light chain (V_{L}) polypeptide comprising
a V_{L} CDR1 comprising the amino acid sequence

| | |
|---|---|
| RASQSVSSAVA, | (SEQ ID NO: 38) |

a V_{L} CDR2 comprising the amino acid sequence

| | |
|---|---|
| SASSLYS, | (SEQ ID NO: 39) |
| and | |

a V_{L} CDR3 comprising the amino acid sequence

| | |
|---|---|
| YYSSPL. | (SEQ ID NO: 40) |

In certain embodiments, an antibody of the present disclosure specifically binds to CD55 and competes for binding to CD55 with an antibody comprising:

a variable heavy chain (V_{H}) polypeptide comprising
(SEQ ID NO: 42)
a V_{H} CDR1 comprising the amino acid sequence
GFTFSSYYIH,
(SEQ ID NO: 43)
a V_{H} CDR2 comprising the amino acid sequence
AAISSSSGSTYYADS, and
(SEQ ID NO: 44)
a V_{H} CDR3 comprising the amino acid sequence
CAREDYYWGDFVFDYWGQG; and
a variable light chain (V_{L}) polypeptide comprising
(SEQ ID NO: 46)
a V_{L} CDR1 comprising the amino acid sequence
RASQSVSSAVA,
(SEQ ID NO: 47)
a V_{L} CDR2 comprising the amino acid sequence
SASSLYS, and
(SEQ ID NO: 48)
a V_{L} CDR3 comprising the amino acid sequence
YYSSPL.

According to some embodiments, an antibody of the present disclosure specifically binds to CD55 and competes for binding to CD55 with an antibody comprising:

a variable heavy chain (V_{H}) polypeptide comprising
(SEQ ID NO: 50)
a V_{H} CDR1 comprising the amino acid sequence
GFTFTDYDIH,
(SEQ ID NO: 51)
a V_{H} CDR2 comprising the amino acid sequence
AGWINPYNGYTDYADS, and
(SEQ ID NO: 52)
a V_{H} CDR3 comprising the amino acid sequence
CARPLSWALDYWGQG; and
a variable light chain (V_{L}) polypeptide comprising
(SEQ ID NO: 54)
a V_{L} CDR1 comprising the amino acid sequence
RASQDVNTAVA,
(SEQ ID NO: 55)
a V_{L} CDR2 comprising the amino acid sequence
SASFLYS, and
(SEQ ID NO: 56)
a V_{L} CDR3 comprising the amino acid sequence
HYTTPP.

In certain embodiments, an antibody of the present disclosure specifically binds to CD55 and competes for binding to CD55 with an antibody comprising:

a variable heavy chain (V_{H}) polypeptide comprising
(SEQ ID NO: 58)
a V_{H} CDR1 comprising the amino acid sequence
GFTFSSYYIH,
(SEQ ID NO: 59)
a V_{H} CDR2 comprising the amino acid sequence
AYIRPSGSKTSYADS, and
(SEQ ID NO: 60)
a V_{H} CDR3 comprising the amino acid sequence
CARKVYARSGLDYWGQG; and
a variable light chain (V_{L}) polypeptide comprising
(SEQ ID NO: 62)
a V_{L} CDR1 comprising the amino acid sequence
RASQSVSSAVA,
(SEQ ID NO: 63)
a V_{L} CDR2 comprising the amino acid sequence
SASSLYS, and
(SEQ ID NO: 64)
a V_{L} CDR3 comprising the amino acid sequence
GESTPY.

According to some embodiments, an antibody of the present disclosure specifically binds to CD55 and competes for binding to CD55 with an antibody comprising:

a variable heavy chain (V_{H}) polypeptide comprising
(SEQ ID NO: 66)
a V_{H} CDR1 comprising the amino acid sequence
GFTFSSYGIH,
(SEQ ID NO: 67)
a V_{H} CDR2 comprising the amino acid sequence
AYIYSRGTVTSYADS, and
(SEQ ID NO: 68)
a V_{H} CDR3 comprising the amino acid sequence
CARGAGGVMDYWGQG; and
a variable light chain (V_{L}) polypeptide comprising
(SEQ ID NO: 70)
a V_{L} CDR1 comprising the amino acid sequence
RASQSVSSAVA,
(SEQ ID NO: 71)
a V_{L} CDR2 comprising the amino acid sequence
SASSLYS, and
(SEQ ID NO: 72)
a V_{L} CDR3 comprising the amino acid sequence
AKSSLR.

Any suitable approach for determining whether a first antibody competes with a second antibody for binding to CD55 may be employed. Non-limiting examples of such approaches include competition ELISA (e.g., as described in Example 4 below), competitive cell binding assays (e.g., as described in Example 5 below), and the like.

Methods are available for measuring the affinity of an anti-CD55 antibody for CD55 expressed on the surface of cells (e.g., T cells) using direct binding or competition binding assays. In a direct binding assay, the equilibrium binding constant (K_{D}) may be measured using a candidate anti-CD55 antibody conjugated to a fluorophore or radioisotope, or a candidate anti-CD55 antibody that contains an N- or C-terminal epitope tag for detection by a labeled antibody. If labels or tags are not feasible or desired, a competition binding assay can be used to determine the half-maximal inhibitory concentration (IC₅₀), the amount of unlabeled candidate anti-CD55 antibody at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

According to some embodiments, an antibody of the present disclosure specifically binds to CD55 and comprises:

a variable heavy chain (V_{H}) polypeptide comprising
(SEQ ID NO: 2)
a V_{H} CDR1 comprising the amino acid sequence
GFTFSSYYIH,
(SEQ ID NO: 3)
a V_{H} CDR2 comprising the amino acid sequence
AISSSSGSTYYADS, and
a V_{H} CDR3 comprising the amino acid sequence
(SEQ ID NO: 4)
CARERYFYGDFVFDYWGQG; and
a variable light chain (V_{L}) polypeptide comprising
(SEQ ID NO: 6)
a V_{L} CDR1 comprising the amino acid sequence
RASQSVSSAVA,
(SEQ ID NO: 7)
a V_{L} CDR2 comprising the amino acid sequence
SASSLYS, and
(SEQ ID NO: 8)
a V_{L} CDR3 comprising the amino acid sequence
YYSSPL.

In certain embodiments, an antibody of the present disclosure specifically binds to CD55 and comprises:

a variable heavy chain (V_{H}) polypeptide comprising
(SEQ ID NO: 10)
a V_{H} CDR1 comprising the amino acid sequence
GFTFSHHYIH,
(SEQ ID NO: 11)
a V_{H} CDR2 comprising the amino acid sequence
AISTGGGTTYYADS, and
(SEQ ID NO: 12)
a V_{H} CDR3 comprising the amino acid sequence
CARERYFNGDFIYDYWGQG; and
a variable light chain (V_{L}) polypeptide comprising
(SEQ ID NO: 14)
a V_{L} CDR1 comprising the amino acid sequence
RASQSVSSAVA,
(SEQ ID NO: 15)
a V_{L} CDR2 comprising the amino acid sequence
SASSLYS, and
(SEQ ID NO: 16)
a V_{L} CDR3 comprising the amino acid sequence
YYSTPL.

According to some embodiments, an antibody of the present disclosure specifically binds to CD55 and comprises:

a variable heavy chain (V_{H}) polypeptide comprising
(SEQ ID NO: 18)
a V_{H} CDR1 comprising the amino acid sequence
GFTFSSYYIH,
(SEQ ID NO: 19)
a V_{H} CDR2 comprising the amino acid sequence
AISSSSGSTYYADS, and
(SEQ ID NO: 20)
a V_{H} CDR3 comprising the amino acid sequence
CAREVRIHGDFVLDYWGQG; and
a variable light chain (V_{L}) polypeptide comprising
(SEQ ID NO: 22)
a V_{L} CDR1 comprising the amino acid sequence
RASQSVSSAVA,
(SEQ ID NO: 23)
a V_{L} CDR2 comprising the amino acid sequence
SASSLYS, and
(SEQ ID NO: 24)
a V_{L} CDR3 comprising the amino acid sequence
YYSSPL.

In certain embodiments, an antibody of the present disclosure specifically binds to CD55 and comprises:

a variable heavy chain (V_{H}) polypeptide comprising
(SEQ ID NO: 26)
a V_{H} CDR1 comprising the amino acid sequence
GFTFSSYYIH,
(SEQ ID NO: 27)
a V_{H} CDR2 comprising the amino acid sequence
AAISSSSGSTYYADS, and
(SEQ ID NO: 28)
a V_{H} CDR3 comprising the amino acid sequence
CAREARFHGDFILDYWGQG; and
a variable light chain (V_{L}) polypeptide comprising
(SEQ ID NO: 30)
a V_{L} CDR1 comprising the amino acid sequence
RASQSVSSAVA,
(SEQ ID NO: 31)
a V_{L} CDR2 comprising the amino acid sequence
SASSLYS, and
(SEQ ID NO: 32)
a V_{L} CDR3 comprising the amino acid sequence
YYITPL.

According to some embodiments, an antibody of the present disclosure specifically binds to CD55 and comprises:

a variable heavy chain (V_{H}) polypeptide comprising
(SEQ ID NO: 34)
a V_{H} CDR1 comprising the amino acid sequence
GFTFSSYYIH,
(SEQ ID NO: 35)
a V_{H} CDR2 comprising the amino acid sequence
AAISSSSGSTYYADS, and
a V_{H} CDR3 comprising the amino acid sequence
(SEQ ID NO: 36)
CAREDWHRYERRFYEGFDYWGQG; and
a variable light chain (V_{L}) polypeptide comprising
(SEQ ID NO: 38)
a V_{L} CDR1 comprising the amino acid sequence
RASQSVSSAVA,
(SEQ ID NO: 39)
a V_{L} CDR2 comprising the amino acid sequence
SASSLYS, and
(SEQ ID NO: 40)
a V_{L} CDR3 comprising the amino acid sequence
YYSSPL.

In certain embodiments, an antibody of the present disclosure specifically binds to CD55 and comprises:

a variable heavy chain (V_{H}) polypeptide comprising
(SEQ ID NO: 42)
a V_{H} CDR1 comprising the amino acid sequence
GFTFSSYYIH,
(SEQ ID NO: 43)
a V_{H} CDR2 comprising the amino acid sequence
AAISSSSGSTYYADS, and
a V_{H} CDR3 comprising the amino acid sequence
(SEQ ID NO: 44)
CAREDYYWGDFVFDYWGQG; and
a variable light chain (V_{L}) polypeptide comprising
(SEQ ID NO: 46)
a V_{L} CDR1 comprising the amino acid sequence
RASQSVSSAVA,
(SEQ ID NO: 47)
a V_{L} CDR2 comprising the amino acid sequence
SASSLYS, and
(SEQ ID NO: 48)
a V_{L} CDR3 comprising the amino acid sequence
YYSSPL.

According to some embodiments, an antibody of the present disclosure specifically binds to CD55 and comprises:

a variable heavy chain (V_{H}) polypeptide comprising
(SEQ ID NO: 50)
a V_{H} CDR1 comprising the amino acid sequence
GFTFTDYDIH,
(SEQ ID NO: 51)
a V_{H} CDR2 comprising the amino acid sequence
AGWINPYNGYTDYADS, and
(SEQ ID NO: 52)
a V_{H} CDR3 comprising the amino acid sequence
CARPLSWALDYWGQG; and
a variable light chain (V_{L}) polypeptide comprising
(SEQ ID NO: 54)
a V_{L} CDR1 comprising the amino acid sequence
RASQDVNTAVA,
(SEQ ID NO: 55)
a V_{L} CDR2 comprising the amino acid sequence
SASFLYS, and
(SEQ ID NO: 56)
a V_{L} CDR3 comprising the amino acid sequence
HYTTPP.

In certain embodiments, an antibody of the present disclosure specifically binds to CD55 and comprises:

a variable heavy chain (V_{H}) polypeptide comprising
(SEQ ID NO: 58)
a V_{H} CDR1 comprising the amino acid sequence
GFTFSSYYIH,
(SEQ ID NO: 59)
a V_{H} CDR2 comprising the amino acid sequence
AYIRPSGSKTSYADS, and
(SEQ ID NO: 60)
a V_{H} CDR3 comprising the amino acid sequence
CARKVYARSGLDYWGQG; and
a variable light chain (V_{L}) polypeptide comprising
(SEQ ID NO: 62)
a V_{L} CDR1 comprising the amino acid sequence
RASQSVSSAVA,
(SEQ ID NO: 63)
a V_{L} CDR2 comprising the amino acid sequence
SASSLYS, and
(SEQ ID NO: 64)
a V_{L} CDR3 comprising the amino acid sequence
GESTPY.

According to some embodiments, an antibody of the present disclosure specifically binds to CD55 and comprises:

a variable heavy chain (V_{H}) polypeptide comprising
(SEQ ID NO: 66)
a V_{H} CDR1 comprising the amino acid sequence
GFTFSSYGIH,
(SEQ ID NO: 67)
a V_{H} CDR2 comprising the amino acid sequence
AYIYSRGTVTSYADS, and
(SEQ ID NO: 68)
a V_{H} CDR3 comprising the amino acid sequence
CARGAGGVMDYWGQG; and
a variable light chain (V_{L}) polypeptide comprising
(SEQ ID NO: 70)
a V_{L} CDR1 comprising the amino acid sequence
RASQSVSSAVA,
(SEQ ID NO: 71)
a V_{L} CDR2 comprising the amino acid sequence
SASSLYS, and
(SEQ ID NO: 72)
a V_{L} CDR3 comprising the amino acid sequence
AKSSLR.

In certain embodiments, an antibody of the present disclosure specifically binds to CD55, competes for binding to CD55 with an antibody comprising one, two, three, four, five, or all six CDRs of the antibody designated herein as P5-1, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:1; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:5.

In some embodiments, such an antibody comprises one, two, three, four, five, or all six CDRs, including any combination thereof, of the antibody designated herein as P5-1.

According to some embodiments, an antibody of the present disclosure specifically binds to CD55, competes for binding to CD55 with an antibody comprising one, two, three, four, five, or all six CDRs of the antibody designated herein as P5-1J, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:9; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:13.

In some embodiments, such an antibody comprises one, two, three, four, five, or all six CDRs, including any combination thereof, of the antibody designated herein as P5-1J.

In certain embodiments, an antibody of the present disclosure specifically binds to CD55, competes for binding to CD55 with an antibody comprising one, two, three, four, five, or all six CDRs of the antibody designated herein as P5-2, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:21.

In some embodiments, such an antibody comprises one, two, three, four, five, or all six CDRs, including any combination thereof, of the antibody designated herein as P5-2.

According to some embodiments, an antibody of the present disclosure specifically binds to CD55, competes for binding to CD55 with an antibody comprising one, two, three, four, five, or all six CDRs of the antibody designated herein as P5-2-2A, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:25; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:29.

In some embodiments, such an antibody comprises one, two, three, four, five, or all six CDRs, including any combination thereof, of the antibody designated herein as P5-2-2A.

In certain embodiments, an antibody of the present disclosure specifically binds to CD55, competes for binding to CD55 with an antibody comprising one, two, three, four, five, or all six CDRs of the antibody designated herein as P5-3, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:33; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:37.

In some embodiments, such an antibody comprises one, two, three, four, five, or all six CDRs, including any combination thereof, of the antibody designated herein as P5-3.

According to some embodiments, an antibody of the present disclosure specifically binds to CD55, competes for binding to CD55 with an antibody comprising one, two, three, four, five, or all six CDRs of the antibody designated herein as P5-4, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:41; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:45.

In some embodiments, such an antibody comprises one, two, three, four, five, or all six CDRs, including any combination thereof, of the antibody designated herein as P5-4.

In certain embodiments, an antibody of the present disclosure specifically binds to CD55, competes for binding to CD55 with an antibody comprising one, two, three, four, five, or all six CDRs of the antibody designated herein as P5-10B, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:49; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:53.

In some embodiments, such an antibody comprises one, two, three, four, five, or all six CDRs, including any combination thereof, of the antibody designated herein as P5-10B.

According to some embodiments, an antibody of the present disclosure specifically binds to CD55, competes for binding to CD55 with an antibody comprising one, two, three, four, five, or all six CDRs of the antibody designated herein as P5-34, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:57; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:61.

In some embodiments, such an antibody comprises one, two, three, four, five, or all six CDRs, including any combination thereof, of the antibody designated herein as P5-34.

In certain embodiments, an antibody of the present disclosure specifically binds to CD55, competes for binding to CD55 with an antibody comprising one, two, three, four, five, or all six CDRs of the antibody designated herein as P5-37, and wherein the antibody comprises:
a variable heavy chain (V_{H}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:65; and
a variable light chain (V_{L}) polypeptide comprising an amino acid sequence having 70% or greater, 75% or greater, 80% or greater, 85% or greater, 90% or greater, 95% or greater, or 99% or greater identity to the amino acid sequence set forth in SEQ ID NO:69.

In some embodiments, such an antibody comprises one, two, three, four, five, or all six CDRs, including any combination thereof, of the antibody designated herein as P5-37.

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In some embodiments, the antibody is selected from an IgG, Fv, single chain antibody, scFv, Fab, F(ab′)₂, or Fab′. The antibodies may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

An immunoglobulin light or heavy chain variable region is composed of a “framework” region (FR) interrupted by three hypervariable regions, also called “complementarity determining regions” or “CDRs”. The extent of the framework region and CDRs can be defined based on databases known in the art. See, for example, “Sequences of Proteins of Immunological Interest,” E. Kabat et al., Sequences of proteins of immunological interest, 4th ed. U.S. Dept. Health and Human Services, Public Health Services, Bethesda, Md. (1987), Lefranc et al. IMGT, the international ImMunoGeneTics Information System®. Nucl. Acids Res., 2005, 33:D593-D597 (www.imgt.org/textes/IMGTScientificChart/), and/or V Base at vbase.mrc-cpe.cam.ac.uk/). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of the antigen in the presence of a heterogeneous population of antigens (e.g., proteins and other biologics, e.g., in a tissue). Thus, under designated immunoassay conditions, the specified antibodies bind to a particular antigen and do not bind in a significant amount to other antigens present in the sample. Specific binding to an antigen under such conditions may require an antibody that is selected for its specificity for a particular antigen. For example, anti-CD55 antibodies can specifically bind to CD55, and do not exhibit comparable binding (e.g., do not exhibit detectable binding) to other proteins present in a tissue sample.

In some embodiments, an antibody of the present disclosure “specifically binds” to CD55 if it binds to or associates with CD55 with an affinity or Kₐ (that is, an equilibrium association constant of a particular binding interaction with units of 1/M) of, for example, greater than or equal to about 10⁵ M⁻¹. In certain embodiments, the antibody binds to CD55 with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to CD55 with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for CD55 can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

An “epitope” is a site on an antigen (e.g., a site on CD55) to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with a membrane associated antigen can reside on the surface of the cell (e.g. in the extracellular region of a transmembrane protein), so that such epitopes are considered cell-surface accessible, solvent accessible, and/or cell-surface exposed.

Also provided are conjugates. The conjugates include an anti-CD55 antibody of the present disclosure, and an agent conjugated to the antibody. The term “conjugated” generally refers to a chemical linkage, either covalent or non-covalent, usually covalent, that proximally associates one molecule of interest with a second molecule of interest. In some embodiments, the agent is selected from a half-life extending moiety, a labeling agent, and a therapeutic agent. For half-life extension, for example, the antibodies of the present disclosure can optionally be modified to provide for improved pharmacokinetic profile (e.g., by PEGylation, hyperglycosylation, and the like). Modifications that can enhance serum half-life are of interest. A subject antibody may be “PEGylated”, as containing one or more poly(ethylene glycol) (PEG) moieties. Methods and reagents suitable for PEGylation of a protein are well known in the art and may be found in U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject protein can be linear. The PEG conjugated to the subject protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody is to be isolated from a source, the subject protein can be conjugated to moieties the facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies are to be detected in an assay, the antibodies may contain a detectable label, e.g., a radioisotope (e.g., ¹²⁵I; ³⁵S, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., ¹⁵²Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

Any of the above elements that are used to modify the subject antibody may be linked to the antibody via a linker, e.g. a flexible linker. If present, the linker molecules are generally of sufficient length to permit the antibody and a linked carrier to allow some flexible movement between the antibody and the carrier. The linker molecules are generally about 6-50 atoms long. The linker molecules may also be, for example, aryl acetylene, ethylene glycol oligomers containing 2-10 monomer units, diamines, diacids, amino acids, or combinations thereof.

Where the linkers are peptide, the linkers can be of any of a suitable of different lengths, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of a peptide conjugated to any elements described above can include linkers that are all or partially flexible, such that the linker can include a flexible linker as well as one or more portions that confer less flexible structure.

According to some embodiments, the antibody is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9(1):33-46; and elsewhere.

Numerous strategies are available for linking agents to an antibody directly, or indirectly via a linker. For example, the agent may be derivatized by covalently attaching a linker to the agent, where the linker has a functional group capable of reacting with a “chemical handle” on the antibody. The functional group on the linker may vary and may be selected based on compatibility with the chemical handle on the antibody. According to one embodiment, the chemical handle on the antibody is provided by incorporation of an unnatural amino acid having the chemical handle into the antibody. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. An unnatural amino acid may be incorporated into an antibody via chemical synthesis or recombinant approaches (e.g., using a suitable orthogonal amino acyl tRNA synthetase-tRNA pair for incorporation of the unnatural amino acid during translation of the antibody in a host cell).

The functional group of an unnatural amino acid present in the antibody may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody and the linker portion of a linker-sialic acid modulator moiety may include an alkyne functional group, such that the antibody and linker-sialic acid modulator moiety are covalently conjugated via azide-alkyne cycloaddition. Conjugation may be carried out using, e.g., a copper-catalyzed azide-alkyne cycloaddition reaction.

In some embodiments, the chemical handle on the antibody does not involve an unnatural amino acid. An antibody containing no unnatural amino acids may be conjugated to the agent by utilizing, e.g., nucleophilic functional groups of the antibody (such as the N-terminal amine or the primary amine of lysine, or any other nucleophilic amino acid residue) as a nucleophile in a substitution reaction with a moiety bearing a reactive leaving group or other electrophilic group. An example would be to prepare a sialic acid modulator-linker or drug-linker moiety bearing an N-hydroxysuccinimidyl (NHS) ester and allow it to react with the antibody under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody to each other may vary depending upon the particular linker, agent/or antibody and functional groups selected and employed for conjugating the various components to each other.

Also provided are fusion proteins. The fusion proteins include a variable heavy chain (V_{H}) polypeptide, a variable light chain (V_{L}) polypeptide, or both, of an antibody of the present disclosure, fused to a heterologous amino acid sequence. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, luciferase, etc.) are said to be “heterologous” to one another. Similarly, a CDR from a mouse antibody and a constant region from a human antibody are said to be “heterologous” to one another.

Methods of Producing Antibodies

Using the information provided herein, the anti-CD55 antibodies of the present disclosure may be prepared using standard techniques well known to those of skill in the art. For example, a nucleic acid sequence encoding the amino acid sequence of an antibody of the present disclosure can be used to express the antibodies. The polypeptide sequences provided herein (see, e.g., Table 1) can be used to determine appropriate nucleic acid sequences encoding the antibodies and the nucleic acids sequences then used to express one or more antibodies specific for CD55. The nucleic acid sequence(s) can be optimized to reflect particular codon “preferences” for various expression systems according to standard methods well known to those of skill in the art.

Using the sequence information provided, the nucleic acids may be synthesized according to a number of standard methods known to those of skill in the art. Oligonucleotide synthesis, is preferably carried out on commercially available solid phase oligonucleotide synthesis machines or manually synthesized using, for example, a solid phase phosphoramidite triester method.

Once a nucleic acid encoding a subject antibody is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies of the present disclosure can be achieved by operably linking a nucleic acid encoding the antibody to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies of the present disclosure may contain modifications to facilitate isolation, as discussed above.

The subject antibodies may be prepared in substantially pure or isolated form (e.g., free from other polypeptides). The protein can be present in a composition that is enriched for the polypeptide relative to other components that may be present (e.g., other polypeptides or other host cell components). Purified antibodies may be provided such that the antibody is present in a composition that is substantially free of other expressed proteins, e.g., less than 90%, usually less than 60% and more usually less than 50% of the composition is made up of other expressed proteins.

The antibodies produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies of the present disclosure, where suitable cells include eukaryotic cells, e.g., mammalian cells. The cells can be a hybrid cell or “hybridoma” that is capable of reproducing antibodies in vitro (e.g. monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia) insect or mammalian expression system, for example. One example of a suitable technique uses a bacteriophage lambda vector system having a leader sequence that causes the expressed antibody (e.g., Fab or scFv) to migrate to the periplasmic space (between the bacterial cell membrane and the cell wall) or to be secreted. One can rapidly generate great numbers of functional fragments (e.g., Fab or scFv) for those which bind the tumor associated antigen.

Antibodies that specifically bind CD55 can be prepared using a wide variety of techniques known in the art including the use of hybridoma, recombinant, phage display technologies, or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds CD55 can be selected or identified with CD55, e.g., using labeled CD55 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol. 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

Bispecific Antibodies

Also provided are bispecific antibodies. According to some embodiments, a bispecific antibody of the present disclosure includes a first antigen-binding domain (e.g., a Fab arm, scFv, or the like) that specifically binds CD55, where the first antigen binding domain includes a V_{H} polypeptide and a V_{L} polypeptide of an antibody of the present disclosure. In certain embodiments, the bispecific antibody includes a second antigen-binding domain (e.g., a Fab arm, scFv, or the like) that specifically binds CD55. According to some embodiments, the bispecific antibody includes a second antigen-binding domain (e.g., a Fab arm, scFv, or the like) that specifically binds an antigen other than CD55. Examples of antigens other than CD55 to which the second antigen-binding domain may specifically bind include, but are not limited to, a cell surface antigen co-expressed with CD55 on the surface of a cell. For example, the second antigen-binding domain may specifically bind a T cell surface antigen. Other examples of antigens other than CD55 to which the second antigen-binding domain may specifically bind include, but are not limited to, a cell surface antigen expressed on the surface of a cell which does not co-express CD55.

Bispecific antibodies of the present disclosure include antibodies having a full length antibody structure, and bispecific antibody fragments. “Full length” as used herein refers to an antibody having two full length antibody heavy chains and two lull length antibody light chains. A full length antibody heavy chain (HC) consists of well known heavy chain variable and constant domains VH, CH1, CH2, and CH3. A full length antibody light chain (LC) consists of well-known light chain variable and constant domains VL and CL. The full length antibody may be lacking the C-terminal lysine in either one or both heavy chains. The term “Fab arm” refers to one heavy chain-light chain pair that specifically binds an antigen.

Full length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., PCT Intl. Publ. No. WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies. heterodimerization may be promoted by following substitutions (expressed as modified position in the first CH3 domain of the first heavy chain/modified position in the second CH3 domain of the second heavy chain): L351 Y_F405A_Y407V T394W, T366I_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in U.S. Pat. Pubi. No. US2012/0149876 or U.S. Pat. Publ. No. US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8(18):3689-3696.

Nucleic Acids, Expression Vectors and Cells

In view of the section above regarding methods of producing the antibodies of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody of the present disclosure. According to some embodiments, the antibody is a single chain antibody (e.g., an scFv), and the nucleic acid encodes the single chain antibody.

Also provided are expression vectors comprising any of the nucleic acids of the present disclosure.

Cells that comprise any of the nucleic acids and/or expression vectors of the present disclosure are also provided. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such a cell comprises a first expression vector comprising the first nucleic acid, and a second expression vector comprising the second nucleic acid.

Also provided are methods of making an antibody of the present disclosure, the methods including culturing a cell of the present disclosure under conditions suitable for the cell to express the antibody, wherein the antibody is produced. The conditions suitable for the cell to express the antibody may vary. Non-limiting examples of such conditions include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., from 3% to 10%, such as 5%.

### Compositions

The present disclosure also provides compositions. In certain embodiments, the compositions find use, e.g., in practicing the methods of the present disclosure.

According to some embodiments, a composition of the present disclosure includes an antibody of the present disclosure. For example, the antibody may be any of the antibodies described in the Antibodies section hereinabove, which is incorporated but not reiterated herein for purposes of brevity. According to some embodiments, a composition of the present disclosure includes a conjugate of the present disclosure. In some embodiments, a composition of the present disclosure includes a fusion protein of the present disclosure. Any of the compositions of the present disclosure may further include a T cell activator and/or innate immune system stimulator.

In certain aspects, a composition of the present disclosure includes the antibody present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

As summarized above, aspects of the present disclosure include pharmaceutical compositions. In some embodiments, a pharmaceutical composition of the present disclosure includes an anti-CD55 antibody of the present disclosure (or conjugate or fusion protein comprising same), and a pharmaceutically acceptable carrier. In certain aspects, a pharmaceutical composition of the present disclosure includes the antibody (or conjugate or fusion protein comprising same), a T cell activator (e.g., any of the T cell activators described herein), and a pharmaceutically acceptable carrier. In certain aspects, a pharmaceutical composition of the present disclosure includes the antibody (or conjugate or fusion protein comprising same), an innate immune system stimulator (e.g., any of the innate immune system stimulators described herein), and a pharmaceutically acceptable carrier.

As will be appreciated, the pharmaceutical compositions of the present disclosure may include any of the agents and features described herein in the Antibodies and Methods sections, which are incorporated but not reiterated in detail herein for purposes of brevity.

When a pharmaceutical composition of the present disclosure includes a T cell activator, in certain aspects, the T cell activator is an immune checkpoint inhibitor. Immune checkpoint inhibitors of interest include, but are not limited to, an agonist of a T cell co-stimulatory receptor, an antagonist of a T cell inhibitory signal, and/or the like. In some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

When a pharmaceutical composition of the present disclosure includes a T cell activator, in certain aspects, the T cell activator is a cytokine. In some embodiments, when a pharmaceutical composition of the present disclosure includes a T cell activator, the T cell activator is an antagonist of an inhibitory immune receptor. In certain aspects, a pharmaceutical composition of the present disclosure includes a combination (that is, two or more) of any of the T cell activators described herein.

The antibody (and optionally, a T cell activator and/or innate immune system stimulator) can be incorporated into a variety of formulations for therapeutic administration. More particularly, the agent(s) (that is, anti-CD55 antibody, and optionally a T cell activator and/or innate immune system stimulator) can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agents for administration to the individual (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the antibody and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the antibody and/or T cell activator, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods

Aspects of the present disclosure include methods comprising administering an anti-CD55 antibody of the present disclosure (or conjugate or fusion protein comprising same) to an individual in need thereof.

In certain embodiments, provided are methods of treating cell proliferative disorders. According to some embodiments, such methods include administering to an individual having a cell proliferative disorder a therapeutically effective amount of an anti-CD55 antibody of the present disclosure, where at the time of the administering, abnormally proliferating cells of the cell proliferative disorder are not suspected of exhibiting overexpression of CD55. By “not suspected of exhibiting overexpression of CD55” is meant that CD55 is not suspected to be expressed at higher levels on the abnormally proliferating cells (e.g., cancer cells) compared to a second cell population (e.g., non-abnormally proliferating (e.g., non-cancer) cells of the same tissue type as the abnormally proliferating cells). In some embodiments, at the time of the administering, it has been determined that abnormally proliferating cells of the cell proliferative disorder do not overexpress CD55. In certain aspects, the methods include making such a determination.

In some embodiments, provided are methods that include administering to an individual having a cell proliferative disorder a therapeutically effective amount of an anti-CD55 antibody of the present disclosure, where the antibody is administered to the individual to enhance a T cell response to abnormally proliferating cells of the cell proliferative disorder.

The methods of the present disclosure are based at least in part on the inventors' surprising discovery that CD55-binding agents enhance T cell responses to abnormally proliferating cells of a cell proliferative disorder (e.g., cancer), independent of whether the abnormally proliferating cells overexpress CD55. Further details may be found in International Patent Application No. PCT/US2018/047356, the disclosure of which is incorporated herein by reference in its entirety for all purposes. As such, in some embodiments, the methods of the present disclosure are methods of enhancing a T cell response to abnormally proliferating cells of a cell proliferative disorder, e.g., cancer. Similarly, aspects of the present disclosure include administering the anti-CD55 antibody to an individual having a cell proliferative disorder, where the purpose of administering the CD55-binding agent is not to induce, but rather to enhance, antibody-dependent cellular cytotoxicity (ADCC). In some embodiments, the methods include administering the anti-CD55 antibody to an individual receiving an antibody therapy, e.g., an individual receiving an antibody therapy meant to treat a cell proliferative disorder (e.g., cancer) by inducing ADCC, where administering the anti-CD55 antibody enhances the ADCC of the antibody therapy.

The anti-CD55 antibody may be administered to any of a variety of subjects. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a cellular proliferative disorder, e.g., cancer.

As summarized above, the individual in need thereof may have a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer. The subject methods may be employed for the treatment of a large variety of cancers by virtue of the enhanced anti-cancer T cell response achieved. In some embodiments, the individual has a cancer suspected of evading the immune system (e.g., effector T cells), e.g., by co-opting one or more immune checkpoint pathways. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC). In some embodiments, the individual has a cancer for which administration of a T cell activator (e.g., an agonist of a co-stimulatory receptor, an immune checkpoint inhibitor (e.g., a CTLA-4 inhibitor, a PD-1 inhibitor, or the like)) to treat the cancer (alone or in combination with second anti-cancer agent) has been approved by the Food and Drug Administration (FDA).

As summarized above, in certain aspects, the individual to whom the anti-CD55 antibody is administered is receiving an antibody therapy. As used herein, “antibody therapy” means that an antibody (which is not the anti-CD55 antibody) will be, has been, and/or is being administered to the individual for a therapeutic purpose. The antibody therapy will vary depending upon the condition of the individual being treated. In some embodiments, the antibody therapy includes the administration of an antibody (e.g., an IgG, such as an IgG1) that specifically binds to an antigen (e.g., a cell surface antigen, such as a protein or non-protein cell surface antigen) on the surface of a cell relevant to the medical condition of the individual. For example, the antibody administered as part of the antibody therapy may bind to an antigen present on the surface of a cell that contributes to the medical condition (e.g., a cancer cell in an individual having cancer), where binding of the antibody to the antigen reduces or abolishes the cell's contribution to the medical condition. In some embodiments, the individual has cancer, and the antibody administered as part of the antibody therapy is selected from Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, lmgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, and a tumor antigen-binding variant thereof. In some embodiments, the antibody of the antibody therapy is an antibody approved by the United States Food and Drug Administration and/or the European Medicines Agency (EMA) for use as a therapeutic antibody (e.g., for targeting certain disease-associated cells in a patient, etc.), or a fragment thereof (e.g., a single-chain version of such an antibody, such as an scFv version of the antibody) that retains the ability to specifically bind the target antigen.

In some embodiments, provided are methods that include administering a combination of the anti-CD55 antibody and a T cell activator. According to such methods, the administering may be to an individual having a cell proliferative disorder. Optionally, at the time of the administering (e.g., the initial administration of the anti-CD55 antibody, the T cell activator, or both (if present in a single formulation)), abnormally proliferating cells of the cell proliferative disorder are not suspected of exhibiting overexpression of CD55.

As used herein, a “T cell activator” is an agent that stimulates an immune response in a T cell or group of T cells. Such an immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell activation and effector function. Following epitope-specific cell targeting, the targeted T cells are activated through engagement of costimulatory proteins found on the APC with counterpart costimulatory proteins on the T cells. Both signals—epitope/TCR binding and engagement of APC costimulatory proteins with T cell costimulatory proteins—are required to drive T cell specificity and activation. The TCR is specific for a given epitope; however, the costimulatory protein is not epitope specific and instead is generally expressed on all T cells or on large T cell subsets.

Various assays can be utilized in order to determine whether an immune response has been stimulated in a T cell or group of T cells, i.e., whether a T cell or group of T cells has become “activated”. In certain aspects, stimulation of an immune response in T cells can be determined by measuring antigen-induced production of cytokines by T cells. In some embodiments, stimulation of an immune response in T cells can be determined by measuring antigen-induced production of IFNγ, IL-4, IL-2, IL-10, IL-17 and/or TNFα by T cells. In some embodiments, antigen-produced production of cytokines by T cells can be measured by intracellular cytokine staining followed by flow cytometry. In some embodiments, antigen-induced production of cytokines by T cells can be measured by surface capture staining followed by flow cytometry. In some embodiments, antigen-induced production of cytokines by T cells can be measured by determining cytokine concentration in supernatants of activated T cell cultures. In some embodiments, this can be measured by ELISA.

In some embodiments, antigen-produced production of cytokines by T cells can be measured by ELISPOT assay. In general, ELISPOT assays employ a technique very similar to the sandwich enzyme-linked immunosorbent assay (ELISA) technique. An antibody (e.g. monoclonal antibody, polyclonal antibody, etc.) is coated aseptically onto a PVDF (polyvinylidene fluoride)-backed microplate. Antibodies are chosen for their specificity for the cytokine in question. The plate is blocked (e.g. with a serum protein that is non-reactive with any of the antibodies in the assay). Cells of interest are plated out at varying densities, along with antigen or mitogen, and then placed in a humidified 37° C. CO₂ incubator for a specified period of time. Cytokine secreted by activated cells is captured locally by the coated antibody on the high surface area PVDF membrane. After washing the wells to remove cells, debris, and media components, a secondary antibody (e.g., a biotinylated polyclonal antibody) specific for the cytokine is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually a blackish blue) typically represents an individual cytokine-producing cell. Spots can be counted manually (e.g., with a dissecting microscope) or using an automated reader to capture the microwell images and to analyze spot number and size. In some embodiments, each spot correlates to a single cytokine-producing cell.

In some instances, T cells activated by the T cell activator are specific for an epitope present on abnormally proliferative cells underlying the cellular proliferative disorder (e.g., cancer cells in an individual having cancer), and contacting such T cells with the T cell activator increases cytotoxic activity of the T cells toward the abnormally proliferating cells, increases the number of such epitope-specific T cells, or a combination thereof.

A wide variety of known agents may be employed as the T cell activator. In some embodiments, the T cell activator is an immune checkpoint inhibitor. As used herein, an “immune checkpoint inhibitor” is any agent (e.g., small molecule, nucleic acid, protein (e.g., antibody)) that prevents the suppression of any component in the immune system such as MHC class presentation, T cell presentation and/or differentiation, any cytokine, chemokine or signaling for immune cell proliferation and/or differentiation. In certain aspects, the immune checkpoint inhibitor is an agonist of a T cell co-stimulatory receptor. Non-limiting examples of such co-stimulatory receptors include CD28, ICOS, CD137, OX40, CD27, and the like. In some embodiments, the immune checkpoint inhibitor is an antagonist of a T cell inhibitory signal. The T cell inhibitory signal may be a signal transmitted through, e.g., PD-1, PD-L1, CTLA4, BTLA, KIR, LAG-3, TIM-3, A2aR, or the like, and any combinations thereof.

In some embodiments, the T cell activator is an immune checkpoint inhibitor selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, a CD40 agonist, and any combination thereof.

In certain aspects, the T cell activator is a cytokine. Cytokines of interest in the context of the present disclosure are those that promote T cell activation (e.g., IL-1, and the like), promote proliferation of activated T cells (e.g., IL-2, and the like), etc. Non-limiting examples of cytokines that may be administered with the anti-CD55 antibody include IL-1, IL-2, IL-4, IL-15, and any combination thereof.

In some embodiments, the T cell activator is an antagonist of an inhibitory immune receptor. In certain aspects, such an antagonist binds directly to the inhibitory immune receptor, thereby blocking activation of the inhibitory immune receptor, e.g., by preventing binding of the receptor to its ligand. In other aspects, such an antagonist binds to the ligand of an inhibitory immune receptor, thereby blocking activation of the inhibitory immune receptor by preventing binding of the receptor to its ligand. Antagonists of inhibitory immune receptors that may be administered with the anti-CD55 antibody include, but are not limited to, TGF-β.

In some embodiments, the anti-CD55 antibody is administered to the individual with an agent that blocks immune suppressive cytokines. In one non-limiting example, such an agent is a TGF-β receptor inhibitor.

In some embodiments, provided are methods that include administering a combination of the anti-CD55 antibody and an innate immune system stimulator. According to such methods, the administering may be to an individual having a cell proliferative disorder. Optionally, at the time of the administering (e.g., the initial administration of the anti-CD55 antibody, the T cell activator, or both (if present in a single formulation)), abnormally proliferating cells of the cell proliferative disorder are not suspected of exhibiting overexpression of CD55.

As used herein, an “innate immune system stimulator” is an agent that stimulates an innate immune system response in the individual. Examples of agents that may be employed to stimulate the innate immune system include, but are not limited to, agents that bind to one or more Toll-like receptors (TLRs), e.g., one or more of TLR1-TLR9. Most mammalian species have 10-13 types of TLRs and each receptor recognizes specific ligands and induces a wide array of inflammatory cascades.

In some embodiments, the innate immune system stimulator is an agent that includes unmethylated CpG dinucleotides. It is now understood that the immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation; cytokine and immunoglobulin secretion; natural killer (NK) cell lytic activity and IFN-γ secretion; and activation of dendritic cells (DCs) and other antigen presenting cells to express costimulatory molecules and secrete cytokines, especially the Th1-like cytokines that are important in promoting the development of Th1-like T cell responses. These immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci USA 96:9305-10).

The anti-CD55 antibody and, if also administered, a T cell activator and/or innate immune system stimulator, are administered in a therapeutically effective amount. By “therapeutically effective amount” is meant a dosage sufficient to produce a desired result, e.g., an amount sufficient to effect beneficial or desired therapeutic (including preventative) results, such as a reduction in a symptom of the proliferative disorder, as compared to a control. When the cell proliferative disorder is cancer, in some embodiments, the therapeutically effective amount is sufficient to slow the growth of a tumor, reduce the size of a tumor, and/or the like. An effective amount can be administered in one or more administrations.

When the methods include administering a combination of the anti-CD55 antibody and a second agent (e.g., a T cell activator and/or innate immune system stimulator), the anti-CD55 antibody and the second agent may be administered concurrently (e.g., in the same or separate formulations), sequentially, or both. For example, according to certain embodiments, the second agent is administered to the individual prior to administration of the anti-CD55 antibody, concurrently with administration of the anti-CD55 antibody, or both. In some embodiments, the anti-CD55 antibody is administered to the individual prior to administration of the second agent, concurrently with administration of the second agent, or both.

In certain aspects, the one or more agents are administered according to a dosing regimen approved for individual use. In some embodiments, the administration of the anti-CD55 antibody permits the second agent to be administered according to a dosing regimen that involves one or more lower and/or less frequent doses, and/or a reduced number of cycles as compared with that utilized when the second agent is administered without administration of the anti-CD55 antibody. In some embodiments, the administration of the second agent permits the anti-CD55 antibody to be administered according to a dosing regimen that involves one or more lower and/or less frequent doses, and/or a reduced number of cycles as compared with that utilized when the anti-CD55 antibody is administered without administration of the second agent.

As noted above, in certain aspects, one or more doses of the anti-CD55 antibody and second agent are administered at the same time; in some such embodiments, such agents may be administered present in the same pharmaceutical composition. In some embodiments, however, the anti-CD55 antibody and second agent are administered to the individual in different compositions and/or at different times. For example, the anti-CD55 antibody may be administered prior to administration of the second agent (e.g., in a particular cycle). Alternatively, the second agent may be administered prior to administration of the anti-CD55 antibody (e.g., in a particular cycle). The second agent to be administered may be administered a period of time that starts at least 1 hour, 3 hours, 6 hours, 12 hours, 24 hours, 48 hours, 72 hours, or up to 5 days or more after the administration of the first agent.

In one example, the anti-CD55 antibody is administered to the individual for a desirable period of time prior to administration of the second agent. In certain aspects, such a regimen “primes” the immune system, e.g., for T cell activation by the T cell activator and/or innate immune system stimulation by the innate immune system stimulator. In another example, the second agent is administered to the individual for a desirable period of time prior to administration of the anti-CD55 antibody. In certain aspects, such a regimen “primes” the immune system for T cell activation by the anti-CD55 antibody.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

By way of example, the anti-CD55 antibody may be administered a period of time after administration of a T cell activator and/or innate immune system stimulator. The period of time may be selected to be correlated with an increase in T cell activation and/or innate immune system stimulation by the T cell activator and/or innate immune system stimulator, respectively. In some embodiments, the relevant period of time permits (e.g., is correlated with) T cell activation and/or innate immune system stimulation that is 10% or more, 20% or more, 30% or more, 40% or more, 50% or more, 60% or more, 70% or more, 80% or more, 90% or more, 100% or more, 150% or more, 200% or more, 250% or more, 300% or more, 350% or more, 400% or more, 450% or more, or 500% or more, than that observed prior to (or at the moment of) the administration of a T cell activator and/or innate immune system stimulator, respectively.

In some embodiments, the anti-CD55 antibody and second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating the effect of the anti-CD55 antibody, a T cell activator, or both), may include: (a) a first dosing period during which a therapeutically effective amount a first agent is administered to an individual; (b) a first resting period; (c) a second dosing period during which a therapeutically effective amount of a second agent and, optionally, a third agent, is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event (e.g., increased T cell activation), then the second resting period's length may be determined on the basis of different factors, separately or in combination. Exemplary such factors may include type and/or stage of a cancer against which the agents are administered; identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The anti-CD55 antibody, and if also administered, a second agent (e.g., a T cell activator and/or innate immune system stimulator), may be administered via a route of administration independently selected from oral, parenteral (e.g., by intravenous, intra-arterial, subcutaneous, intramuscular, or epidural injection), topical, or nasal administration. According to certain embodiments, the anti-CD55 antibody and a second agent are both administered parenterally, either concurrently (in the same pharmaceutical composition or separate pharmaceutical compositions) or sequentially.

As described above, aspects of the present disclosure include methods for treating a cell proliferative disorder (e.g., cancer). By treatment is meant at least an amelioration of one or more symptoms associated with the cell proliferative disorder of the individual, where amelioration is used in a broad sense to refer to at least a reduction in the magnitude of a parameter, e.g. symptom, associated with the cell proliferative disorder (e.g., cancer) being treated. As such, treatment also includes situations where the cell proliferative disorder, or at least one or more symptoms associated therewith, are completely inhibited, e.g., prevented from happening, or stopped, e.g., terminated, such that the individual no longer suffers from the cell proliferative disorder, or at least the symptoms that characterize the cell proliferative disorder.

### Kits

As summarized above, the present disclosure provides kits. The kits find use, e.g., in practicing the methods of the present disclosure. In some embodiments, a subject kit includes a pharmaceutical composition that includes an anti-CD55 antibody of the present disclosure (e.g., any of the CD55-binding agents described elsewhere herein). In certain aspects, provided are kits that include any of the pharmaceutical compositions described herein, including any of the pharmaceutical compositions described above in the section relating to the compositions of the present disclosure. Kits of the present disclosure may include instructions for administering the pharmaceutical composition to an individual in need thereof, including but not limited to, an individual having a cell proliferative disorder, e.g., cancer. In some embodiments, a kit of the present disclosure includes instructions for administering the pharmaceutical composition to an individual having a cell proliferative disorder in which abnormally proliferating cells of the cell proliferative disorder are not suspected of exhibiting overexpression of CD55. In some embodiments, a kit of the present disclosure includes instructions for administering the pharmaceutical composition to an individual having a cell proliferative disorder to enhance a T cell response to abnormally proliferating cells of the cell proliferative disorder. In certain aspects, a kit of the present disclosure includes instructions for administering the pharmaceutical composition to an individual receiving an antibody therapy. In some embodiments, the antibody therapy is being administered to the individual to treat the cell proliferative disorder by inducing antibody-dependent cellular cytotoxicity (ADCC), and the instructions are for administering the anti-CD55 antibody to the individual to potentiate ADCC of the antibody therapy.

In some embodiments, kits are provided that include an anti-CD55 antibody of the present disclosure, a T cell activator, and instructions for administering the anti-CD55 antibody and T cell activator to an individual having a cell proliferative disorder, e.g., cancer. The instructions may further include any of the instructions for the kits described above, e.g., for administering the pharmaceutical composition to an individual having a cell proliferative disorder to enhance a T cell response to abnormally proliferating cells of the cell proliferative disorder (e.g., cancer), for administering the pharmaceutical composition to an individual receiving an antibody therapy, and/or the like. The anti-CD55 antibody and T cell activator may be present in the same container, or may be present in separate containers.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes an anti-CD55 antibody of the present disclosure, a T cell activator, or both. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular anti-CD55 antibody and/or T cell activator employed, the effect to be achieved, and the pharmacodynamics associated with the anti-CD55 antibody and/or T cell activator, in the individual. In yet other embodiments, the kits may include a single multi dosage amount of the composition.

As will be appreciated, the kits of the present disclosure may include any of the agents and features described above in the sections relating to the subject methods and compositions, which are not reiterated in detail herein for purposes of brevity.

When a kit of the present disclosure includes a T cell activator, in certain aspects, the T cell activator is an immune checkpoint inhibitor. Immune checkpoint inhibitors of interest include, but are not limited to, an agonist of a T cell co-stimulatory receptor, an antagonist of a T cell inhibitory signal, and/or the like. In some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist. When a kit of the present disclosure includes a T cell activator, in certain aspects, the T cell activator is a cytokine. In some embodiments, when a kit of the present disclosure includes a T cell activator, the T cell activator is an antagonist of an inhibitory immune receptor. In certain aspects, a kit of the present disclosure includes a combination (that is, two or more) of any of the T cell activators described herein.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Identification of Human Antibodies that Bind Both Mouse and Human CD55

Human antibody libraries were screened for binding to human and mouse CD55 with emphasis on antibodies that bind both mouse and human CD55. Two parent antibodies were found to bind both mouse and human CD55, and five parent antibodies were found to bind only human CD55. P5-1J is an affinity-matured form of the parent Ab P5-1. P5-2-2A was derived by mutation of the parent Ab P5-2 to obtain binding to mouse and human CD55. A summary of the results is provided in Table 2 below.

TABLE 2
Human Anti-CD55 Antibodies

Species specificity

| Antibody | Mouse CD55 | Human CD55 |
|---|---|---|
| P5-1 | X | X |
| P5-1J* | X | X |
| P5-2 | | X |
| P5-2-2A** | X | X |
| P5-3 | | X |
| P5-4 | X | X |
| P5-10B | | X |
| P5-34 | | X |
| P5-37 | | |

*Affinity maturation of P5-1
**Mutation of P5-2 to achieve recognition of mouse CD55

### Example 2—Co-Stimulation of Human CD4 T-Cell Proliferation

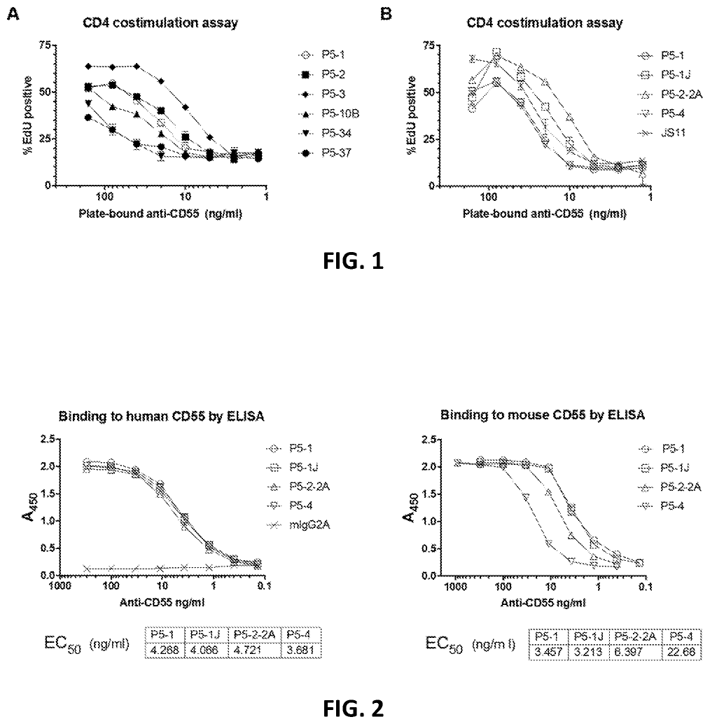

The antibodies described in Example 1 were tested for function based on their ability to co-stimulate human CD4 T-cell proliferation in combination with subthreshold anti-CD3 antibody where all antibodies are plate-bound. Results are shown in FIG. 1. Panel A: costimulatory activity of antibodies P5-1, P5-2, P5-3, P5-10B P5-34, P5-37. Panel B: Costimulatory activity of P5-1, P5-1J, P5-2-2A, P5-4. Solid symbols represent the antibodies that recognize only human CD55, and open symbols represent antibodies that recognized both mouse and human CD55. The commercially available anti-CD55 antibody, JS11, was included in panel B as a positive control. Each antibody co-stimulated CD4 T-cell proliferation to varying degrees. The differential biologic effectiveness of the P5-1, P5-1J, P5-2-2A and P5-4 antibodies in this assay was not reflected by their relative affinity (see FIG. 2, panel A).

### Example 3—Relative Binding Affinities for Mouse and Human CD55

The antibodies that bind both mouse and human CD55 (P5-1, P5-1J, P5-2-2A and P5-4) were analyzed by ELISA for their relative affinities to human CD55 (FIG. 2, panel A) and mouse CD55 (FIG. 2, panel B). EC₅₀ values were obtained by fitting to a sigmoid non-linear equation, using PRISM. These human antibodies contained mouse IgG2A Fc domains for use in in vivo mouse tumor models and were detected by HRP-anti-mouse secondary antibodies. The antibodies tested had very similar affinities for human CD55 (about 4 ng/ml) but varied affinities for mouse CD55 (about 3-22 ng/ml).

### Example 4—Epitope Comparison for Antibodies that Bind Both Mouse and Human CD55 by Competition ELISA

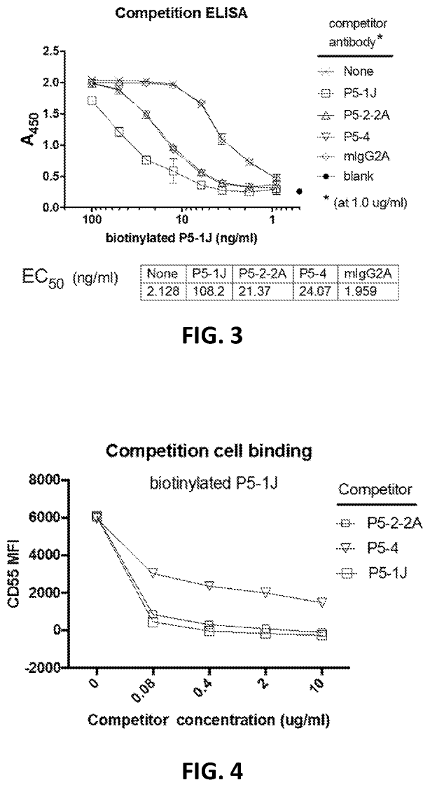

Comparison of the epitopes of the mouse+human CD55-binding antibodies was performed by competition ELISA. Since P5-1 and P5-1J share their epitope, P5-1 was not examined. Plate-bound CD55 was incubated with unlabeled P5-1J, P5-2-2A and P5-4 antibodies at 1 pg/ml. The plates were washed and then incubated with the indicated concentrations of biotinylated P5-1J. Streptavidin-HRP was used for detection of bound P5-1J. Results are shown in FIG. 3. The EC₅₀ values are shown below the graph. P5-2-2A and P5-4 blocked biotinylated P5-1J binding (20-fold shift in EC₅₀) but not to the extent that unlabeled P5-1J blocked it (100-fold shift in EC₅₀). The data suggests that the P5-1J, P5-2-2A and P5-4 epitopes are the same, overlapping, or near enough to cause steric interference.

### Example 5—Epitope Comparison for Antibodies that Bind Both Mouse and Human CD55 by Competitive Cell Binding

Comparison of the epitopes of the mouse+human CD55-binding antibodies was performed by competitive cell binding. Results are shown in FIG. 4. Human peripheral blood mononuclear cells (PBMCs) were incubated with the concentrations of P5 antibodies indicated on the abscissa, washed and then labeled with biotinylated P5-1J followed by Streptavidin-PE and finally analyzed by flow cytometry. The mean fluorescence intensity (MFI) is a measure of the amount of biotinylated P5-1J bound to the cells. Binding to cells is more biologically relevant than binding to purified protein. Both P5-2-2A and P5-4 significantly reduced the binding of P5-1J to CD55, this time on cells and consistent with the ELISA results indicating either identity or close proximity of the P5-1J, P5-2-2A and P5-4 antibody epitopes on CD55.

### Example 6—Effect of Anti-CD55 Antibodies on Tumor Growth In Vivo

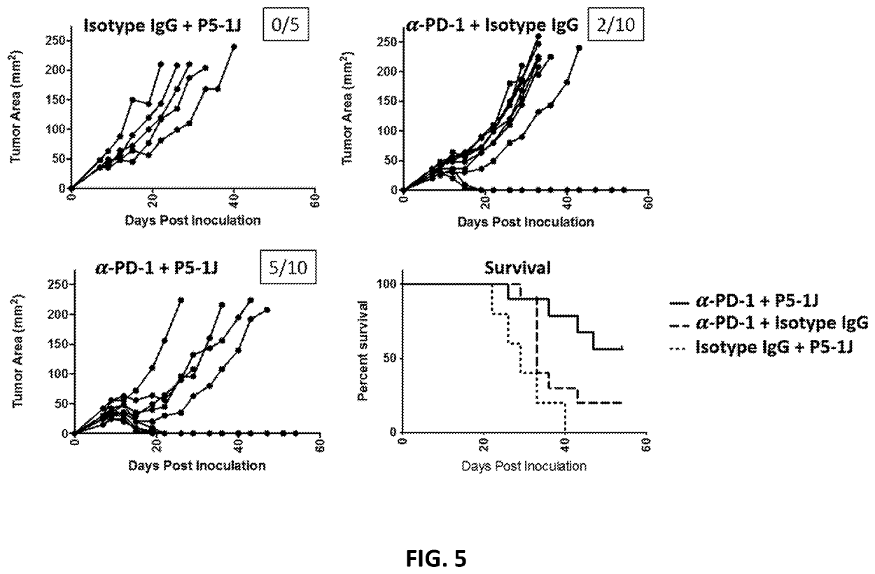

In a first study, mice were inoculated s.c. with 4e5 MCA 205 tumor cells on Day 0. Tumor sizes were measured 2-3 times per week. Mice were treated on days 9, 12 and 15 with either 200 μg of hamster IgG control antibody or 200 μg anti-PD1 i.p. concurrently with either 200 μg of mouse IgG control antibody or antibody P5-1J i.p.. Results are shown in FIG. 5. Single agent anti-PD-1-treated mice had 2/10 mice reject their tumors. The anti-PD-1 and P5-1J combination treatment group had 5/10 mice reject their tumors. Spider plots are shown for each treatment group, where each line represents a single mouse. A survival curve is shown in the lower right corner.

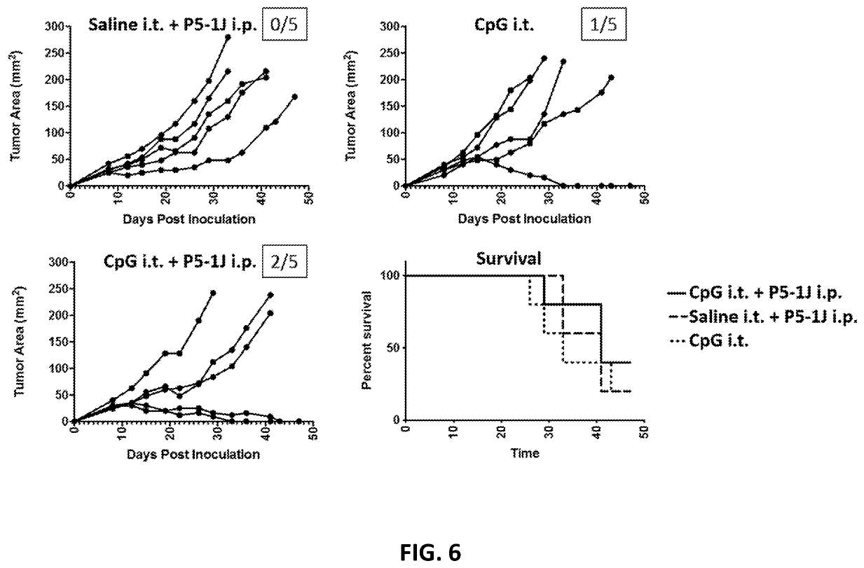

In a second study, mice were inoculated s.c. with 4e5 MCA 205 tumor cells on Day 0. Tumor sizes were measured 2-3 times per week. Mice were treated on days 12, 14 and 16 with either 10 μl of saline i.t. or 30 μg of CpG i.t. alone or concurrently with 200 μg of antibody P5-1J i.p.. Results are shown in FIG. 6. Single therapeutic agent CpG-treated mice had 1/5 mice reject their tumors. The CpG and P5-1J combination treatment group had 2/5 mice reject their tumors. Spider plots are shown for each treatment group, each line represents a single mouse. A survival curve is shown in the lower right corner.

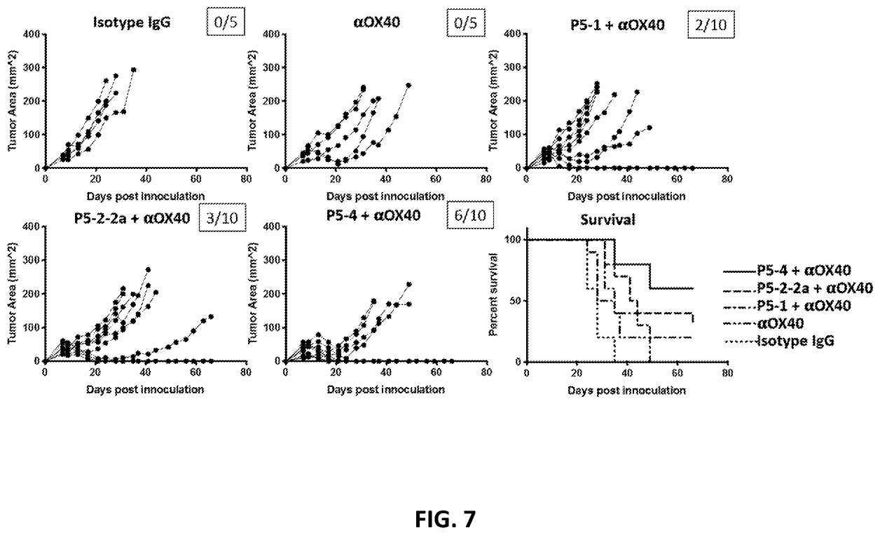

In a further study, mice were inoculated s.c. with 4e5 MCA 205 tumor cells on Day 0. Tumor sizes were measured 2-3 times per week. Mice were treated on days 7, 9 and 11 with either 200 μg of isotype control antibody or 250 μg anti-OX40 i.p. concurrently with either 200 μg of mouse IgG control antibody or antibody P5-1, P5-2-2a, or P5-4 i.p.. Results are shown in FIG. 7. None of the isotype control antibody-treated mice rejected their tumor. Single therapeutic agent anti-OX40-treated mice had 0/5 mice reject their tumors. The P5-1 and anti-OX40 combination treatment group had 2/10 mice reject their tumors. The P5-2-2a and anti-OX40 combination treatment group had 3/10 mice reject their tumors. The P5-4 and anti-OX40 combination treatment group had 6/10 mice reject their tumors. Spider plots are shown for each treatment group, each line represents a single mouse. A survival curve is shown in the lower right corner.

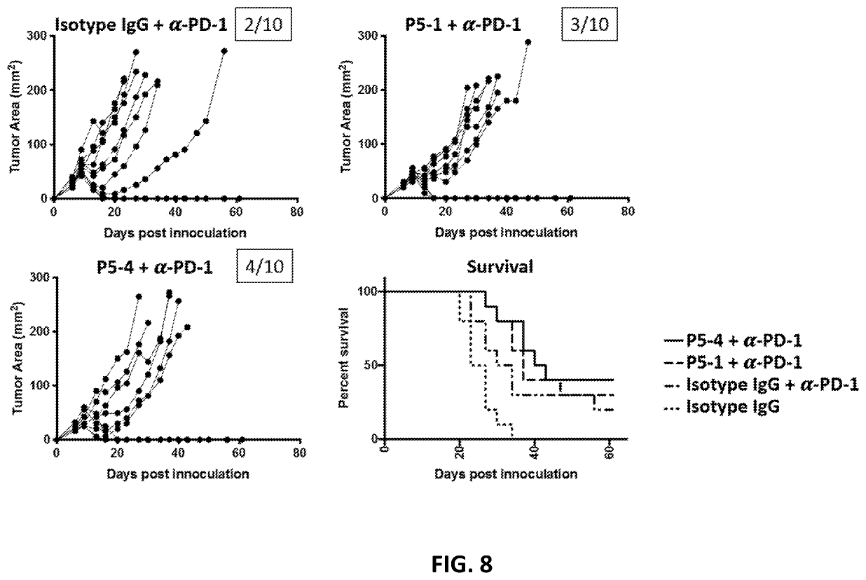

In a further study, mice were inoculated s.c. with 4e5 MCA 205 tumor cells on Day 0. Tumor sizes were measured 2-3 times per week. Mice were treated on days 6, 8 and 10 with either 200 μg of isotype control antibody or 200 μg anti-PD-1 i.p. concurrently with either 200 μg of mouse IgG control antibody or antibody P5-1 or P5-4 i.p.. Results are shown in FIG. 8. None of the isotype control antibody-treated mice rejected their tumor. Single therapeutic agent anti-PD-1-treated mice had 2/10 mice reject their tumors. The P5-1 and anti-PD-1 combination treatment group had 3/10 mice reject their tumors. The P5-4 and anti-PD-1 combination treatment group had 4/10 mice reject their tumors. Spider plots are shown for each treatment group (except the isotype only group), each line represents a single mouse. A survival curve is shown in the lower right corner.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An antibody that specifically binds to CD55 and competes for binding to CD55 with an antibody comprising:
a variable heavy chain (V_{H}) polypeptide comprising
(SEQ ID NO: 2)
a V_{H} CDR1 comprising the amino acid sequence GFTFSSYYIH,
(SEQ ID NO: 3)
a V_{H} CDR2 comprising the amino acid sequence AISSSSGSTYYADS,
and
a V_{H} CDR3 comprising the amino acid sequence
(SEQ ID NO: 4)
CARERYFYGDFVFDYWGQG; and
a variable light chain (V_{L}) polypeptide comprising
(SEQ ID NO: 6)
a V_{L} CDR1 comprising the amino acid sequence RASQSVSSAVA,
(SEQ ID NO: 7)
a V_{L} CDR2 comprising the amino acid sequence SASSLYS,
and
(SEQ ID NO: 8)
a V_{L} CDR3 comprising the amino acid sequence YYSSPL;
a variable heavy chain (V_{H}) polypeptide comprising
(SEQ ID NO: 10)
a V_{H} CDR1 comprising the amino acid sequence GFTFSHHYIH,
(SEQ ID NO: 11)
a V_{H} CDR2 comprising the amino acid sequence AISTGGGTTYYADS,
and
(SEQ ID NO: 12)
a V_{H} CDR3 comprising the amino acid sequence CARERYFNGDFIYDYWGQG;
and
a variable light chain (V_{L}) polypeptide comprising
(SEQ ID NO: 14)
a V_{L} CDR1 comprising the amino acid sequence RASQSVSSAVA,
(SEQ ID NO: 15)
a V_{L} CDR2 comprising the amino acid sequence SASSLYS,
and
(SEQ ID NO: 16)
a V_{L} CDR3 comprising the amino acid sequence YYSTPL;
a variable heavy chain (V_{H}) polypeptide comprising
(SEQ ID NO: 18)
a V_{H} CDR1 comprising the amino acid sequence GFTFSSYYIH,
(SEQ ID NO: 19)
a V_{H} CDR2 comprising the amino acid sequence AISSSSGSTYYADS,
and
(SEQ ID NO: 20)
a V_{H} CDR3 comprising the amino acid sequence CAREVRIHGDFVLDYWGQG;
and
a variable light chain (V_{L}) polypeptide comprising
(SEQ ID NO: 22)
a V_{L} CDR1 comprising the amino acid sequence RASQSVSSAVA,
(SEQ ID NO: 23)
a V_{L} CDR2 comprising the amino acid sequence SASSLYS,
and
(SEQ ID NO: 24)
a V_{L} CDR3 comprising the amino acid sequence YYSSPL;
a variable heavy chain (V_{H}) polypeptide comprising
(SEQ ID NO: 26)
a V_{H} CDR1 comprising the amino acid sequence GFTFSSYYIH,
(SEQ ID NO: 27)
a V_{H} CDR2 comprising the amino acid sequence AAISSSSGSTYYADS,
and
(SEQ ID NO: 28)
a V_{H} CDR3 comprising the amino acid sequence CAREARFHGDFILDYWGQG;
and
a variable light chain (V_{L}) polypeptide comprising
(SEQ ID NO: 30)
a V_{L} CDR1 comprising the amino acid sequence RASQSVSSAVA,
(SEQ ID NO: 31)
a V_{L} CDR2 comprising the amino acid sequence SASSLYS,
and
(SEQ ID NO: 32)
a V_{L} CDR3 comprising the amino acid sequence YYITPL;
a variable heavy chain (V_{H}) polypeptide comprising
(SEQ ID NO: 34)
a V_{H} CDR1 comprising the amino acid sequence GFTFSSYYIH,
(SEQ ID NO: 35)
a V_{H} CDR2 comprising the amino acid sequence AAISSSSGSTYYADS,
and
a V_{H} CDR3 comprising the amino acid sequence
(SEQ ID NO: 36)
CAREDWHRYERRFYEGFDYWGQG; and
a variable light chain (V_{L}) polypeptide comprising,
(SEQ ID NO: 38)
a V_{L} CDR1 comprising the amino acid sequence RASQSVSSAVA,
(SEQ ID NO: 39)
a V_{L} CDR2 comprising the amino acid sequence SASSLYS,
and
(SEQ ID NO: 40)
a V_{L} CDR3 comprising the amino acid sequence YYSSPL;
a variable heavy chain (V_{H}) polypeptide comprising
(SEQ ID NO: 42)
a V_{H} CDR1 comprising the amino acid sequence GFTFSSYYIH,
(SEQ ID NO: 43)
a V_{H} CDR2 comprising the amino acid sequence AAISSSSGSTYYADS,
and
a V_{H} CDR3 comprising the amino acid sequence
(SEQ ID NO: 44)
CAREDYYWGDFVFDYWGQG; and
a variable light chain (V_{L}) polypeptide comprising
(SEQ ID NO: 46)
a V_{L} CDR1 comprising the amino acid sequence RASQSVSSAVA,
(SEQ ID NO: 47)
a V_{L} CDR2 comprising the amino acid sequence SASSLYS,
and
(SEQ ID NO: 48)
a V_{L} CDR3 comprising the amino acid sequence YYSSPL;
a variable heavy chain (V_{H}) polypeptide comprising
(SEQ ID NO: 50)
a V_{H} CDR1 comprising the amino acid sequence GFTFTDYDIH,
(SEQ ID NO: 51)
a V_{H} CDR2 comprising the amino acid sequence AGWINPYNGYTDYADS,
and
(SEQ ID NO: 52)
a V_{H} CDR3 comprising the amino acid sequence CARPLSWALDYWGQG;
and
a variable light chain (V_{L}) polypeptide comprising
(SEQ ID NO: 54)
a V_{L} CDR1 comprising the amino acid sequence RASQDVNTAVA,
(SEQ ID NO: 55)
a V_{L} CDR2 comprising the amino acid sequence SASFLYS,
and
(SEQ ID NO: 56)
a V_{L} CDR3 comprising the amino acid sequence HYTTPP;
a variable heavy chain (V_{H}) polypeptide comprising
(SEQ ID NO: 58)
a V_{H} CDR1 comprising the amino acid sequence GFTFSSYYIH,
(SEQ ID NO: 59)
a V_{H} CDR2 comprising the amino acid sequence AYIRPSGSKTSYADS,
and
(SEQ ID NO: 60)
a V_{H} CDR3 comprising the amino acid sequence CARKVYARSGLDYWGQG;
and
a variable light chain (V_{L}) polypeptide comprising
(SEQ ID NO: 62)
a V_{L} CDR1 comprising the amino acid sequence RASQSVSSAVA,
(SEQ ID NO: 63)
a V_{L} CDR2 comprising the amino acid sequence SASSLYS,
and
(SEQ ID NO: 64)
a V_{L} CDR3 comprising the amino acid sequence GESTPY; or
a variable heavy chain (V_{H}) polypeptide comprising
(SEQ ID NO: 66)
a V_{H} CDR1 comprising the amino acid sequence GFTFSSYGIH,
(SEQ ID NO: 67)
a V_{H} CDR2 comprising the amino acid sequence AYIYSRGTVTSYADS,
and
(SEQ ID NO: 68)
a V_{H} CDR3 comprising the amino acid sequence CARGAGGVMDYWGQG;
and
a variable light chain (V_{L}) polypeptide comprising
(SEQ ID NO: 70)
a V_{L} CDR1 comprising the amino acid sequence RASQSVSSAVA,
(SEQ ID NO: 71)
a V_{L} CDR2 comprising the amino acid sequence SASSLYS,
and
(SEQ ID NO: 72)
a V_{L} CDR3 comprising the amino acid sequence AKSSLR.

2. The antibody of claim 1, wherein the antibody comprises:
a variable heavy chain (V_{H}) polypeptide comprising
(SEQ ID NO: 2)
a V_{H} CDR1 comprising the amino acid sequence GFTFSSYYIH,
(SEQ ID NO: 3)
a V_{H} CDR2 comprising the amino acid sequence AISSSSGSTYYADS,
and
a V_{H} CDR3 comprising the amino acid sequence
(SEQ ID NO: 4)
CARERYFYGDFVFDYWGQG; and
a variable light chain (V_{L}) polypeptide comprising
(SEQ ID NO: 6)
a V_{L} CDR1 comprising the amino acid sequence RASQSVSSAVA,
(SEQ ID NO: 7)
a V_{L} CDR2 comprising the amino acid sequence SASSLYS,
and
(SEQ ID NO: 8)
a V_{L} CDR3 comprising the amino acid sequence YYSSPL;
a variable heavy chain (V_{H}) polypeptide comprising
(SEQ ID NO: 10)
a V_{H} CDR1 comprising the amino acid sequence GFTFSHHYIH,
(SEQ ID NO: 11)
a V_{H} CDR2 comprising the amino acid sequence AISTGGGTTYYADS,
and
(SEQ ID NO: 12)
a V_{H} CDR3 comprising the amino acid sequence CARERYFNGDFIYDYWGQG;
and
a variable light chain (V_{L}) polypeptide comprising
(SEQ ID NO: 14)
a V_{L} CDR1 comprising the amino acid sequence RASQSVSSAVA,
(SEQ ID NO: 15)
a V_{L} CDR2 comprising the amino acid sequence SASSLYS,
and
(SEQ ID NO: 16)
a V_{L} CDR3 comprising the amino acid sequence YYSTPL;
a variable heavy chain (V_{H}) polypeptide comprising
(SEQ ID NO: 18)
a V_{H} CDR1 comprising the amino acid sequence GFTFSSYYIH,
(SEQ ID NO: 19)
a V_{H} CDR2 comprising the amino acid sequence AISSSSGSTYYADS,
and
(SEQ ID NO: 20)
a V_{H} CDR3 comprising the amino acid sequence CAREVRIHGDFVLDYWGQG;
and
a variable light chain (V_{L}) polypeptide comprising
(SEQ ID NO: 22)
a V_{L} CDR1 comprising the amino acid sequence RASQSVSSAVA,
(SEQ ID NO: 23)
a V_{L} CDR2 comprising the amino acid sequence SASSLYS,
and
(SEQ ID NO: 24)
a V_{L} CDR3 comprising the amino acid sequence YYSSPL;
a variable heavy chain (V_{H}) polypeptide comprising
(SEQ ID NO: 26)
a V_{H} CDR1 comprising the amino acid sequence GFTFSSYYIH,
(SEQ ID NO: 27)
a V_{H} CDR2 comprising the amino acid sequence AAISSSSGSTYYADS,
and
(SEQ ID NO: 28)
a V_{H} CDR3 comprising the amino acid sequence CAREARFHGDFILDYWGQG;
and
a variable light chain (V_{L}) polypeptide comprising
(SEQ ID NO: 30)
a V_{L} CDR1 comprising the amino acid sequence RASQSVSSAVA,
(SEQ ID NO: 31)
a V_{L} CDR2 comprising the amino acid sequence SASSLYS,
and
(SEQ ID NO: 32)
a V_{L} CDR3 comprising the amino acid sequence YYITPL;
a variable heavy chain (V_{H}) polypeptide comprising
(SEQ ID NO: 34)
a V_{H} CDR1 comprising the amino acid sequence GFTFSSYYIH,
(SEQ ID NO: 35)
a V_{H} CDR2 comprising the amino acid sequence AAISSSSGSTYYADS,
and
a V_{H} CDR3 comprising the amino acid sequence
(SEQ ID NO: 36)
CAREDWHRYERRFYEGFDYWGQG; and
a variable light chain (V_{L}) polypeptide comprising
(SEQ ID NO: 38)
a V_{L} CDR1 comprising the amino acid sequence RASQSVSSAVA,
(SEQ ID NO: 39)
a V_{L} CDR2 comprising the amino acid sequence SASSLYS,
and
(SEQ ID NO: 40)
a V_{L} CDR3 comprising the amino acid sequence YYSSPL;
a variable heavy chain (V_{H}) polypeptide comprising
(SEQ ID NO: 42)
a V_{H} CDR1 comprising the amino acid sequence GFTFSSYYIH,
(SEQ ID NO: 43)
a V_{H} CDR2 comprising the amino acid sequence AAISSSSGSTYYADS,
and
a V_{H} CDR3 comprising the amino acid sequence
(SEQ ID NO: 44)
CAREDYYWGDFVFDYWGQG; and
a variable light chain (V_{L}) polypeptide comprising
(SEQ ID NO: 46)
a V_{L} CDR1 comprising the amino acid sequence RASQSVSSAVA,
(SEQ ID NO: 47)
a V_{L} CDR2 comprising the amino acid sequence SASSLYS,
and
(SEQ ID NO: 48)
a V_{L} CDR3 comprising the amino acid sequence YYSSPL;
a variable heavy chain (V_{H}) polypeptide comprising
(SEQ ID NO: 50)
a V_{H} CDR1 comprising the amino acid sequence GFTFTDYDIH,
(SEQ ID NO: 51)
a V_{H} CDR2 comprising the amino acid sequence AGWINPYNGYTDYADS,
and
(SEQ ID NO: 52)
a V_{H} CDR3 comprising the amino acid sequence CARPLSWALDYWGQG;
and
a variable light chain (V_{L}) polypeptide comprising
(SEQ ID NO: 54)
a V_{L} CDR1 comprising the amino acid sequence RASQDVNTAVA,
(SEQ ID NO: 55)
a V_{L} CDR2 comprising the amino acid sequence SASFLYS,
and
(SEQ ID NO: 56)
a V_{L} CDR3 comprising the amino acid sequence HYTTPP;
a variable heavy chain (V_{H}) polypeptide comprising
(SEQ ID NO: 58)
a V_{H} CDR1 comprising the amino acid sequence GFTFSSYYIH,
(SEQ ID NO: 59)
a V_{H} CDR2 comprising the amino acid sequence AYIRPSGSKTSYADS,
and
(SEQ ID NO: 60)
a V_{H} CDR3 comprising the amino acid sequence CARKVYARSGLDYWGQG;
and
a variable light chain (V_{L}) polypeptide comprising
(SEQ ID NO: 62)
a V_{L} CDR1 comprising the amino acid sequence RASQSVSSAVA,
(SEQ ID NO: 63)
a V_{L} CDR2 comprising the amino acid sequence SASSLYS,
and
(SEQ ID NO: 64)
a V_{L} CDR3 comprising the amino acid sequence GESTPY; or
a variable heavy chain (V_{H}) polypeptide comprising
(SEQ ID NO: 66)
a V_{H} CDR1 comprising the amino acid sequence GFTFSSYGIH,
(SEQ ID NO: 67)
a V_{H} CDR2 comprising the amino acid sequence AYIYSRGTVTSYADS,
and
(SEQ ID NO: 68)
a V_{H} CDR3 comprising the amino acid sequence CARGAGGVMDYWGQG;
and
a variable light chain (V_{L}) polypeptide comprising
(SEQ ID NO: 70)
a V_{L} CDR1 comprising the amino acid sequence RASQSVSSAVA,
(SEQ ID NO: 71)
a V_{L} CDR2 comprising the amino acid sequence SASSLYS,
and
(SEQ ID NO: 72)
a V_{L} CDR3 comprising the amino acid sequence AKSSLR.

3. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:1; and
a variable light chain (V_{L}) polypeptide comprising an amino acid sequence having 70% or greater identity to the amino acid sequence set forth in SEQ ID NO:5.

4. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:9; and
a variable light chain (V_{L}) polypeptide comprising an amino acid sequence having 70% or greater identity to the amino acid sequence set forth in SEQ ID NO:13.

5. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:17; and
a variable light chain (V_{L}) polypeptide comprising an amino acid sequence having 70% or greater identity to the amino acid sequence set forth in SEQ ID NO:21.

6. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:25; and
a variable light chain (V_{L}) polypeptide comprising an amino acid sequence having 70% or greater identity to the amino acid sequence set forth in SEQ ID NO:29.

7. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:33; and
a variable light chain (V_{L}) polypeptide comprising an amino acid sequence having 70% or greater identity to the amino acid sequence set forth in SEQ ID NO:37.

8. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:41; and
a variable light chain (V_{L}) polypeptide comprising an amino acid sequence having 70% or greater identity to the amino acid sequence set forth in SEQ ID NO:45.

9. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:49; and
a variable light chain (V_{L}) polypeptide comprising an amino acid sequence having 70% or greater identity to the amino acid sequence set forth in SEQ ID NO:53.

10. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:57; and
a variable light chain (V_{L}) polypeptide comprising an amino acid sequence having 70% or greater identity to the amino acid sequence set forth in SEQ ID NO:61.

11. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO:65; and
a variable light chain (V_{L}) polypeptide comprising an amino acid sequence having 70% or greater identity to the amino acid sequence set forth in SEQ ID NO:69.

12. The antibody of any one of claims 1 to 11, wherein the antibody is a humanized antibody.

13. The antibody of any one of claims 1 to 12, wherein the antibody is selected from the group consisting of: an IgG, Fv, single chain antibody, scFv, Fab, F(ab′)2, or Fab′.

14. The antibody of any one of claims 1 to 12, wherein the antibody is an IgG.

15. The antibody of claim 14, wherein the antibody is an IgG1.

16. The antibody of any one of claims 1 to 12, wherein the antibody is a Fab.

17. The antibody of any one of claims 1 to 12, wherein the antibody is a single chain antibody.

18. The antibody of claim 17, wherein the antibody is an scFv.

19. The antibody of any one of claims 1 to 18, wherein the antibody is a bispecific antibody comprising a first antigen-binding domain that specifically binds CD55, and wherein the first antigen binding domain comprises a V_{H} polypeptide-V_{L} polypeptide pair as defined in any one of claims 1 to 11.

20. The antibody of claim 19, wherein the bispecific antibody comprises a second antigen-binding domain that specifically binds CD55.

21. The antibody of claim 19, wherein the bispecific antibody comprises a second antigen-binding domain that specifically binds an antigen other than CD55.

22. The antibody of claim 21, wherein the antigen other than CD55 is a T cell surface antigen.

23. A nucleic acid encoding a variable heavy chain (V_{H}) polypeptide, a variable light chain (V_{L}) polypeptide, or both, of the antibody of any one of claims 1 to 22.

24. The nucleic acid of claim 23, wherein the antibody is a single chain antibody, and wherein the nucleic acid encodes the single chain antibody.

25. The nucleic acid of claim 24, wherein the single chain antibody is an scFv.

26. An expression vector comprising the nucleic acid of any one of claims 23 to 25.

27. A cell comprising the nucleic acid of any one of claims 23 to 25 or the expression vector of claim 26.

28. The cell of claim 27, wherein the nucleic acid encodes the V_{H} polypeptide of the antibody and the V_{L} polypeptide of the antibody.

29. The cell of claim 28, wherein the antibody is a single chain antibody, and wherein the nucleic acid encodes the single chain antibody.

30. The cell of claim 29, wherein the single chain antibody is an scFv.

31. A cell comprising:
a first nucleic acid encoding a variable heavy chain (V_{H}) polypeptide of the antibody of any one of claims 1 to 22; and
a second nucleic acid encoding a variable light chain (V_{L}) polypeptide of the antibody.

32. The cell of claim 31 comprising:
a first expression vector comprising the first nucleic acid; and
a second expression vector comprising the second nucleic acid.

33. A method of making the antibody of any one of claims 1 to 22, comprising culturing the cell of any one of claims 27 to 32 under conditions suitable for the cell to express the antibody, wherein the antibody is produced.

34. A conjugate, comprising:
the antibody of any one of claims 1 to 22; and
an agent conjugated to the antibody.

35. The conjugate of claim 34, wherein the agent is selected from the group consisting of: a half-life extending moiety, a labeling agent, and a therapeutic agent.

36. A fusion protein, comprising:
a variable heavy chain (V_{H}) polypeptide, a variable light chain (V_{L}) polypeptide, or both, of the antibody of any one of claims 1 to 22; fused to
a heterologous amino acid sequence.

37. A pharmaceutical composition, comprising:
the antibody of any one of claims 1 to 22; and
a pharmaceutically acceptable carrier.

38. A pharmaceutical composition, comprising:
the conjugate of claim 34 or 35; and
a pharmaceutically acceptable carrier.

39. A pharmaceutical composition, comprising:
the fusion protein of claim 36; and
a pharmaceutically acceptable carrier.

40. The pharmaceutical composition of any one of claims 37 to 39, further comprising a T cell activator.

41. The pharmaceutical composition of claim 40, wherein the T cell activator is selected from the group consisting of: an immune checkpoint inhibitor, a cytokine, and an antagonist of an inhibitory immune receptor.

42. The pharmaceutical composition of claim 40, wherein the T cell activator is an immune checkpoint inhibitor.

43. The pharmaceutical composition of claim 42, wherein the immune checkpoint inhibitor is an agonist of a T cell co-stimulatory receptor.

44. The pharmaceutical composition of claim 42, wherein the immune checkpoint inhibitor is an antagonist of a T cell inhibitory signal.

45. The pharmaceutical composition of claim 42, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

46. The pharmaceutical composition of claim 40, wherein the T cell activator is a cytokine.

47. The pharmaceutical composition of claim 40, wherein the T cell activator is an antagonist of an inhibitory immune receptor.

48. The pharmaceutical composition of any one of claims 37 to 47, further comprising an innate immune system stimulator.

49. The pharmaceutical composition of claim 48, wherein the innate immune system stimulator is an agent that binds to a Toll-like receptor (TLR).

50. The pharmaceutical composition of claim 49, wherein the agent comprises unmethylated CpG dinucleotides.

51. A kit, comprising:
the pharmaceutical composition of any one of claims 37 to 50; and
instructions for administering the pharmaceutical composition to an individual in need thereof.

52. The kit of claim 51, wherein the pharmaceutical composition is present in one or more unit dosages.

53. The kit of claim 51 or 52, further comprising a T cell activator.

54. The kit of claim 53, wherein the T cell activator is selected from the group consisting of: an immune checkpoint inhibitor, a cytokine, and an antagonist of an inhibitory immune receptor.

55. The kit of claim 53, wherein the T cell activator is an immune checkpoint inhibitor.

56. The kit of claim 55, wherein the immune checkpoint inhibitor is an agonist of a T cell co-stimulatory receptor.

57. The kit of claim 55, wherein the immune checkpoint inhibitor is an antagonist of a T cell inhibitory signal.

58. The kit of claim 55, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

59. The kit of claim 53, wherein the T cell activator is a cytokine.

60. The kit of claim 53, wherein the T cell activator is an antagonist of an inhibitory immune receptor.

61. The kit of any one of claims 51 to 60, further comprising an innate immune system stimulator.

62. The kit of claim 61, wherein the innate immune system stimulator is an agent that binds to a Toll-like receptor (TLR).

63. The kit of claim 62, wherein the agent comprises unmethylated CpG dinucleotides.

64. The kit of any one of claims 51 to 60, wherein the individual in need thereof has a cell proliferative disorder.

65. The kit of claim 64, comprising instructions for administering the pharmaceutical composition to enhance a T cell response to abnormally proliferating cells of the cell proliferative disorder.

66. The kit of any one of claims 51 to 65, comprising instructions for administering the pharmaceutical composition to an individual having a cell proliferative disorder in which abnormally proliferating cells of the cell proliferative disorder are not suspected of exhibiting overexpression of CD55.

67. A method of treating a cell proliferative disorder, comprising:
administering to an individual having a cell proliferative disorder a therapeutically effective amount of the antibody of any one of claims 1 to 22,
wherein at the time of the administering, abnormally proliferating cells of the cell proliferative disorder are not suspected of exhibiting overexpression of CD55.

68. The method according to claim 67, wherein at the time of the administering, it has been determined that abnormally proliferating cells of the cell proliferative disorder do not overexpress CD55.

69. The method according to claim 68, comprising determining that abnormally proliferating cells of the cell proliferative disorder do not overexpress CD55.

70. The method according to any one of claims 67 to 69, wherein the individual to whom the antibody is administered is receiving an antibody therapy.

71. The method according to claim 70, wherein the antibody therapy is being administered to the individual to treat the cell proliferative disorder by inducing antibody-dependent cellular cytotoxicity (ADCC), and wherein the antibody is administered to the individual to potentiate ADCC of the antibody therapy.

72. A method of treating a cell proliferative disorder, comprising:
administering to an individual having a cell proliferative disorder a therapeutically effective amount of the antibody of any one of claims 1 to 22,
wherein the antibody is administered to the individual to enhance a T cell response to abnormally proliferating cells of the cell proliferative disorder.

73. The method according to claim 72, wherein the individual to whom the antibody is administered is receiving an antibody therapy.

74. The method according to claim 73, wherein the antibody therapy is being administered to the individual to treat the cell proliferative disorder by inducing antibody-dependent cellular cytotoxicity (ADCC), and wherein the antibody is administered to the individual to potentiate the ADCC of the antibody therapy.

75. The method according to any one of claims 67 to 74, further comprising administering to the individual a T cell activator.

76. The method according to claim 75, wherein the T cell activator is an immune checkpoint inhibitor.

77. The method according to claim 76, wherein the immune checkpoint inhibitor is an agonist of a T cell co-stimulatory receptor.

78. The method according to claim 76, wherein the immune checkpoint inhibitor is an antagonist of a T cell inhibitory signal.

79. The method according to claim 76, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

80. The method according to claim 75, wherein the T cell activator is a cytokine.

81. The method according to claim 75, wherein the T cell activator is an antagonist of an inhibitory immune receptor.

82. The method according to any one of claims 75 to 81, wherein the antibody and the T cell activator are administered concurrently.

83. The method according to any one of claims 75 to 81, wherein the antibody and the T cell activator are administered sequentially.

84. The method according to any one of claims 67 to 83, further comprising administering to the individual an innate immune system stimulator.

85. The method according to claim 84, wherein the innate immune system stimulator is an agent that binds to a Toll-like receptor (TLR).

86. The method according to claim 85, wherein the agent comprises unmethylated CpG dinucleotides.

87. The method according to any one of claims 67 to 86, wherein the cell proliferative disorder is cancer.

88. A method of treating a cell proliferative disorder, comprising:
administering to an individual having a cell proliferative disorder:
a therapeutically effective amount of the antibody of any one of claims 1 to 22; and
a therapeutically effective amount of a T cell activator.

89. The method according to claim 88, wherein at the time of the administering, abnormally proliferating cells of the cell proliferative disorder are not suspected of exhibiting overexpression of CD55.

90. The method according to claim 89, wherein at the time of the administering, it has been determined that abnormally proliferating cells of the cell proliferative disorder do not overexpress CD55.

91. The method according to claim 90, comprising determining that abnormally proliferating cells of the cell proliferative disorder do not overexpress CD55.

92. The method according to any one of claims 88 to 91, wherein the individual to whom the antibody is administered is receiving an antibody therapy.

93. The method according to claim 92, wherein the antibody therapy is being administered to the individual to treat the cell proliferative disorder by inducing antibody-dependent cellular cytotoxicity (ADCC), and wherein the antibody is administered to the individual to potentiate ADCC of the antibody therapy.

94. The method according to any one of claims 88 to 93, wherein the antibody is administered to the individual to enhance a T cell response to abnormally proliferating cells of the cell proliferative disorder.

95. The method according to any one of claims 88 to 94, wherein the T cell activator is an immune checkpoint inhibitor.

96. The method according to claim 95, wherein the immune checkpoint inhibitor is an agonist of a T cell co-stimulatory receptor.

97. The method according to claim 95, wherein the immune checkpoint inhibitor is an antagonist of a T cell inhibitory signal.

98. The method according to claim 95, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

99. The method according to any one of claims 88 to 94, wherein the T cell activator is a cytokine.

100. The method according to any one of claims 88 to 94, wherein the T cell activator is an antagonist of an inhibitory immune receptor.

101. The method according to any one of claims 88 to 100, wherein the antibody and the T cell activator are administered concurrently.

102. The method according to any one of claims 88 to 100, wherein the antibody and the T cell activator are administered sequentially.

103. The method according to any one of claims 88 to 102, wherein the cell proliferative disorder is cancer.
